(19) United States
(12) Patent Application Publication
(10) Pub. No.: US 2022/0110977 A1
(43) Pub. Date: Apr. 14, 2022

(54) COMPOSITIONS AND METHODS FOR TREATING AND PREVENTING VIRAL INFECTIONS

(71) Applicants: Reza Saadat Niaki Moghaddam, Pacific Grove, CA (US); Jayesh Arvind Desai, Carmel, CA (US)

(72) Inventors: Reza Saadat Niaki Moghaddam, Pacific Grove, CA (US); Jayesh Arvind Desai, Carmel, CA (US)

(21) Appl. No.: 17/556,650

(22) Filed: Dec. 20, 2021

Related U.S. Application Data
(63) Continuation-in-part of application No. 17/407,400, filed on Aug. 20, 2021, now abandoned.
(60) Provisional application No. 63/068,454, filed on Aug. 21, 2020.

(51) Int. Cl. A61K 35/26; A61K 36/481; A61K 35/28; A61K 36/9066; A61K 35/20; A61K 31/375; A61K 36/07; A61K 36/254; A61K 36/185; A61K 36/74; A61K 38/40; A61K 36/484; A61K 36/63; A61K 36/19; A61K 36/81; A61K 35/644; A61K 36/074; A61K 36/738; A61K 31/715; A61K 31/706; A61K 38/38; A61K 36/28; A61K 36/04; A61K 36/537; A61K 36/14; A61K 31/716; A61K 31/355; A61K 36/489; A61K 36/53; A61K 31/4045; A61K 33/06; A61K 31/7076; A61K 35/76; A61K 31/01; A61K 31/593; A61K 31/07; A61K 33/04; A61K 31/714; A61K 45/06; A61K 33/30; A61K 38/47; A61K 38/17; A61P 37/04

(52) U.S. Cl. CPC A61K 35/26; A61P 37/04; A61K 35/28; A61K 36/9066; A61K 35/20; A61K 31/375; A61K 36/07; A61K 36/254; A61K 36/185; A61K 36/74; A61K 38/40; A61K 36/484; A61K 36/63; A61K 36/19; A61K 36/81; A61K 35/644; A61K 36/074; A61K 36/738; A61K 31/715; A61K 31/706; A61K 38/385; A61K 36/28; A61K 36/04; A61K 36/537; A61K 36/14; A61K 31/716; A61K 31/355; A61K 36/489; A61K 36/53; A61K 31/4045; A61K 33/06; A61K 31/7076; A61K 35/76; A61K 31/01; A61K 31/593; A61K 31/07; A61K 33/04; A61K 31/714; A61K 45/06; A61K 33/30; A61K 38/47; C12Y 302/01017; A61K 38/1709; A61K 36/481

(57) ABSTRACT

A pretreatment herbal composition comprises Andrographis paniculata, Eleuthero roots, and resistant potato starch. A treatment herbal composition comprises cannabidiol, bee pollen, and coconut oil, and in some embodiments, additionally comprises hemp seed oil. A fortifying herbal composition comprises many ingredients. A method comprises the steps of providing a dose of apitoxin at least once a day for at least one day, providing a dose of a glucosamine product at least once a day for at least one day, and providing a dose of a treatment herbal composition at least once a day for at least one day. In some embodiments, the method additionally comprises the step of providing a dose of a pretreatment herbal composition at least once a day for at least one day. In further embodiments, the method additionally comprises the step of providing a dose of a fortifying herbal composition.

### CROSS-REFERENCE TO RELATED APPLICATIONS

This application claims the priority benefit of U.S. Provisional Patent Application Ser. No. 63/068,454, filed Aug. 21, 2020, which is herein incorporated by reference in its entirety. As a continuation-in-part (CIP), this application claims the priority of U.S. Non-Provisional patent application Ser. No. 17/407,400, filed on Aug. 20, 2021, which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of immunology, and more specifically to the field of therapeutic and preventative methods for combatting viral infections.

### BACKGROUND

The 2020 global COVID-19 crisis demonstrated that the world needs increased access to preventative and therapeutic means to improve resistance to viruses. While pharmaceuticals indeed offer powerful solutions to human health concerns, they must be administered properly to achieve the desired results. Unfortunately, due to the process of drug discovery and the healthcare system of some notable markets, pharmaceuticals can be prohibitively expensive to some consumers. Moreover, pharmaceuticals are often developed to treat or mitigate deficient health conditions after the patient has acquired the illness or deficiency. Consumers seeking preventative opportunities for bolstering or improving upon their health before contracting an illness often have few options if they limit themselves to the modern pharmaceutical industry and its products.

Traditional medicines can fill this void in the market. Though less immediately effective than modern pharmaceuticals, some believe that these alternatives can provide health benefits that can compound over time if consumed regularly, particularly towards the realm of preventative care. This makes traditional medicines a potentially valuable addition or supplement to modern medical treatments, and these medicines are often cheaper and more accessible than pharmaceutical alternatives.

### SUMMARY

There is a need for new and useful system and method for treating or preventing a viral infection in human patients. In some embodiments, one aspect of the disclosure herein includes for a pretreatment herbal composition for treating or preventing a viral infection in a human patient comprising Andrographis paniculata, Eleuthero roots, and resistant potato starch. In some embodiments, the pretreatment herbal composition comprises about 10% to about 90% Andrographis paniculata, about 10% to about 90% Eleuthero roots, and about 5% to about 50% resistant potato starch by weight percentage. In other embodiments, the pretreatment herbal composition comprises about 20% to about 80% Andrographis paniculata, about 20% to about 80% Eleuthero roots, and about 10% to about 40% resistant potato starch by weight percentage. In further embodiments, the pretreatment herbal composition comprises about 40% Andrographis paniculata, about 40% Eleuthero roots, and about 20% resistant potato starch by weight percentage.

In other embodiments, one aspect of the disclosure herein includes for a treatment herbal composition for treating or preventing a viral infection in a human patient comprising cannabidiol, bee pollen, and coconut oil. In some embodiments, the treatment herbal composition further comprises hemp seed oil. In additional embodiments, the treatment herbal composition comprises about 30% to 90% cannabidiol, about 0.5% to about 25% hemp seed oil, about 5% to 30% bee pollen, and about 15% to 60% coconut oil by weight percentage. In some embodiments, the treatment herbal composition comprises about 30% to 90% cannabidiol, about 1% to about 10% hemp seed oil, about 5% to 15% bee pollen, and about 15% to 45% coconut oil by weight percentage. In further embodiments, the treatment herbal composition comprises about 58% cannabidiol, about 2% hemp seed oil, about 10% bee pollen, and about 30% coconut oil by weight percentage.

In other embodiments, one aspect of the disclosure herein includes for a fortifying herbal composition comprising bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

In some embodiments, the fortifying herbal composition comprises the ingredients by the following proportions by weight percentage: the bovine thymus, Astragalus roots, bovine red bone marrow, and turmeric are each about 5% to about 20%, the bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%, the beta nicotinamide mononucleotide, bovine serum albumin, Echinacea pallida roots, Echinacea purpurea, red marine algae, rosemary leaf, and Thuja occidentals leaf are each about 0.5 to about 5%, the beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 2%, and the lycopene, vitamin D3, vitamin A, selenium, and vitamin B12 are each about 0.0005% to about 0.3%.

In further embodiments, the fortifying herbal composition comprises the ingredients by the following proportions by weight percentage: the bovine thymus, Astragalus roots, bovine red bone marrow, and turmeric are each about 5% to about 10%, the bovine colostrum, bovine immunoglobulin, and vitamin C are each about 3% to about 8%, the turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%, the beta nicotinamide mononucleotide, bovine serum albumin, Echinacea pallida roots, Echinacea purpurea, red marine algae, rosemary leaf, and Thuja occidentals leaf are each about 1% to about 3%, the beta-glucan, transfer factor, umcka, vitamin E, wild indigo roots, oregano, and zinc are each about 0.3% to about 1.5%, the lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 1%, and the lycopene, vitamin D3, vitamin A, selenium, and vitamin B12 are each about 0.0005% to about 0.1%.

In still further embodiments, the fortifying herbal composition comprises the ingredients by the following proportions by weight percentage: the bovine thymus is about 8.693%, the Astragalus roots are about 8.196%, the bovine red bone marrow is about 8.072%, the turmeric is about 7.451%, the bovine colostrum is about 5.588%, the bovine immunoglobulin and vitamin C are each about 4.967%, the turkey tail mushrooms are about 3.726%, the Eleuthera roots and Sambucus nigra are each about 3.105%, the cats claw bark, lactoferrin, licorice, and the olive leaf are each about 2.484%, the Adhatoda vasica, ashwagandha, bee glue, reishi mushrooms, and shiitake mushrooms are each about 2.235%, the rose hip is about 1.987%, the arabinogalactan is about 1.739%, the beta nicotinamide mononucleotide, bovine serum albumin, Echinacea pallida roots, Echinacea purpurea, red marine algae, rosemary leaf, and Thuja occidentals leaf are each about 1.242%, the beta-glucan and transfer factor are each about 0.993%, the umcka, vitamin E, and wild indigo roots are each about 0.745%, the oregano and zinc are each about 0.621%, the lysozyme is about 0.497%, the bee pollen is about 0.373%, the melatonin is about 0.298%, the bentonite clay and royal jelly are each about 0.248%, the adenosine triphosphate, LH01-Myoviridae, LL12-Myoviridae, LL15-Siphoviridae, and T4D-Myroviridae are each about 0.124%, the lycopene is about 0.099%, the vitamin D3 is about 0.072%, the vitamin A is about 0.025%, the selenium is about 0.005%, and the vitamin B12 is about 0.001%.

In still other embodiments, one aspect of the disclosure herein includes for a method for treating or preventing a viral infection in a human patient comprising the steps of: providing a dose of apitoxin to the patient at least once a day for at least one day; providing a dose of glucosamine product to the patient at least once a day for at least one day; providing a dose of a treatment herbal composition to the patient at least once a day for at least one day, wherein the treatment herbal composition comprises cannabidiol, bee pollen, and coconut oil; and wherein the one or more doses of the glucosamine product and the one or more doses of the treatment herbal composition are provided on the same one or more days as the one or more doses of apitoxin.

In some embodiments, the dose of apitoxin is provided for at least 4 consecutive days. In other embodiments, the dose of apitoxin is provided for six consecutive days. In some embodiments, the dose of apitoxin is about 50 to 750 μg per day. In other embodiments, the dose of apitoxin is at least about 200 μg per day. In still other embodiments, the dose of apitoxin increases over the days. In further embodiments, the dose of apitoxin on the first day is about 200 μg, the dose of apitoxin on the second and third days is about 300 μg, and the dose of apitoxin for all remaining subsequent days is about 500 μg. In some embodiments, the dose of apitoxin is provided via parenteral injection. In other embodiments, the dose of apitoxin is provided via subcutaneous injection.

In some embodiments, the glucosamine product is glucosamine sulfate. In other embodiments, the dose of glucosamine sulfate is about 250 mg to 1500 mg. In further embodiments, the dose of glucosamine sulfate is 500 mg and is provided twice a day.

In some embodiments, the treatment herbal composition further comprises hemp seed oil. In additional embodiments, the treatment herbal composition comprises about 30% to 90% cannabidiol, about 0.5% to about 25% hemp seed oil, about 5% to 30% bee pollen, and about 15% to 60% coconut oil by weight percentage. In other embodiments, the treatment herbal composition comprises about 30% to 90% cannabidiol, about 1% to about 10% hemp seed oil, about 5% to 15% bee pollen, and about 15% to 45% coconut oil by weight percentage. In further embodiments, the treatment herbal composition comprises about 58% cannabidiol, about 2% hemp seed oil, about 10% bee pollen, and about 30% coconut oil by weight percentage. In some embodiments, the treatment herbal composition is formulated as a dry powder packaged in a pharmaceutically acceptable capsule for oral delivery. In other embodiments, the pharmaceutically acceptable capsule comprises a vegan gelatin substance.

In some embodiments, the dose of the treatment herbal composition is about 250 to 1500 mg. In other embodiments, the dose of the treatment herbal composition is 500 mg and is provided twice a day. In some embodiments, the at least one dose of the glucosamine product and the at least one dose of the treatment herbal composition are provided on at least each day a dose of apitoxin is provided to the patient. In further embodiments, the at least one dose of glucosamine product and the at least one dose of the treatment herbal composition are taken postprandially.

In some embodiments, the method comprises an additional step of providing a dose of a pretreatment herbal mixture to the patient at least once a day for at least one day, wherein the pretreatment herbal composition comprises Andrographis paniculata, Eleuthero roots, and resistant potato starch. In some embodiments, the pretreatment herbal composition comprises about 10% to about 90% Andrographis paniculata, about 10% to about 90% Eleuthero roots, and about 5% to about 50% resistant potato starch by weight percentage. In other embodiments, the pretreatment herbal composition comprises about 20% to about 80% Andrographis paniculata, about 20% to about 80% Eleuthero roots, and about 10% to about 40% resistant potato starch by weight percentage. In further embodiments, the pretreatment herbal composition comprises about 40% Andrographis paniculata, about 40% Eleuthero roots, and about 20% resistant potato starch by weight percentage. In some embodiments, the pretreatment herbal composition is formulated as a dry powder packaged in a pharmaceutically acceptable capsule for oral delivery. In other embodiments, the pharmaceutically acceptable capsule comprises a vegan gelatin substance.

In some embodiments, the dose of the pretreatment herbal composition is about 300 mg to about 9 grams. In other embodiments, the dose of the pretreatment herbal composition is 1000 mg and wherein the dose of the pretreatment herbal composition is provided three times a day. In some embodiments, the dose of the pretreatment herbal composition is provided for two to seven days. In other embodiments, the dose of the pretreatment herbal composition is provided for four to five days. In some embodiments, at least 24 hours pass between a last dose of the pretreatment herbal composition and a first dose of the apitoxin. In other embodiments, at least 48 hours pass between a last dose of the pretreatment herbal composition and a first dose of the apitoxin.

In some embodiments, the method further comprises the step of providing a dose of a fortifying herbal composition to the patient at least once a day for at least one day. For example, the fortifying herbal composition may include bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

In some embodiments of the method, the fortifying herbal composition comprises the ingredients by the following proportions by weight percentage: the bovine thymus, Astragalus roots, bovine red bone marrow, and turmeric are each about 5% to about 20%, the bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%, the beta nicotinamide mononucleotide, bovine serum albumin, Echinacea pallida roots, Echinacea purpurea, red marine algae, rosemary leaf, and Thuja occidentals leaf are each about 0.5 to about 5%, the beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 2%, and the lycopene, vitamin D3, vitamin A, selenium, and vitamin B12 are each about 0.0005% to about 0.3%.

In some embodiments of the method, the fortifying herbal composition comprises the ingredients by the following proportions by weight percentage: the bovine thymus, Astragalus roots, bovine red bone marrow, and turmeric are each about 5% to about 10%, the bovine colostrum, bovine immunoglobulin, and vitamin C are each about 3% to about 8%, the turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%, the beta nicotinamide mononucleotide, bovine serum albumin, Echinacea pallida roots, Echinacea purpurea, red marine algae, rosemary leaf, and Thuja occidentals leaf are each about 1% to about 3%, the beta-glucan, transfer factor, umcka, vitamin E, wild indigo roots, oregano, and zinc are each about 0.3% to about 1.5%, the lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 1%, and the lycopene, vitamin D3, vitamin A, selenium, and vitamin B12 are each about 0.0005% to about 0.1%.

In some embodiments of the method, the fortifying herbal composition comprises the ingredients by the following proportions by weight percentage: the bovine thymus is about 8.693%, the Astragalus roots are about 8.196%, the bovine red bone marrow is about 8.072%, the turmeric is about 7.451%, the bovine colostrum is about 5.588%, the bovine immunoglobulin and vitamin C are each about 4.967%, the turkey tail mushrooms are about 3.726%, the Eleuthera roots and Sambucus nigra are each about 3.105%, the cats claw bark, lactoferrin, licorice, and the olive leaf are each about 2.484%, the Adhatoda vasica, ashwagandha, bee glue, reishi mushrooms, and shiitake mushrooms are each about 2.235%, the rose hip is about 1.987%, the arabinogalactan is about 1.739%, the beta nicotinamide mononucleotide, bovine serum albumin, Echinacea pallida roots, Echinacea purpurea, red marine algae, rosemary leaf, and Thuja occidentals leaf are each about 1.242%, the beta-glucan and transfer factor are each about 0.993%, the umcka, vitamin E, and wild indigo roots are each about 0.745%, the oregano and zinc are each about 0.621%, the lysozyme is about 0.497%, the bee pollen is about 0.373%, the melatonin is about 0.298%, the bentonite clay and royal jelly are each about 0.248%, the adenosine triphosphate, LH01-Myoviridae, LL12-Myoviridae, LL15-Siphoviridae, and T4D-Myroviridae are each about 0.124%, the lycopene is about 0.099%, the vitamin D3 is about 0.072%, the vitamin A is about 0.025%, the selenium is about 0.005%, and the vitamin B12 is about 0.001%.

In some embodiments of the method, the fortifying herbal composition is formulated as a dry powder packaged in a pharmaceutically acceptable capsule for oral delivery. In other embodiments, the pharmaceutically acceptable capsule comprises a vegan gelatin substance.

In some embodiments of the method, the dose of the fortifying herbal composition is about 300 mg to about 9 grams. In other embodiments, the dose of the pretreatment fortifying herbal composition is 1000 mg and wherein the dose of the pretreatment herbal composition is provided four times a day. In still other embodiments, the dose of the fortifying herbal composition is provided postprandially. In further embodiments, the dose of the fortifying herbal composition is provided for at least two weeks. In still further embodiments, the dose of the fortifying herbal composition is provided for at least four weeks.

One aspect of the disclosure herein includes for, in some embodiments, a fortifying herbal composition comprising: bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

In some embodiments for the fortifying herbal composition, by weight percentage: the bovine thymus, Astragalus roots, bovine red bone marrow, and turmeric are each about 5% to about 20%; the bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%; the beta nicotinamide mononucleotide, bovine serum albumin, Echinacea pallida roots, Echinacea purpurea, red marine algae, rosemary leaf, and Thuja occidentals leaf are each about 0.5 to about 5%; the beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 2%; and the lycopene, vitamin D3, vitamin A, selenium, and vitamin B12 are each about 0.0005% to about 0.3%.

In other embodiments for the fortifying herbal composition, by weight percentage: the bovine thymus, Astragalus roots, bovine red bone marrow, and turmeric are each about 5% to about 10%; the bovine colostrum, bovine immunoglobulin, and vitamin C are each about 3% to about 8%, the turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%; the beta nicotinamide mononucleotide, bovine serum albumin, Echinacea pallida roots, Echinacea purpurea, red marine algae, rosemary leaf, and Thuja occidentals leaf are each about 1% to about 3%; the beta-glucan, transfer factor, umcka, vitamin E, wild indigo roots, oregano, and zinc are each about 0.3% to about 1.5%; the lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 1%; and the lycopene, vitamin D3, vitamin A, selenium, and vitamin B12 are each about 0.0005% to about 0.1%.

In further embodiments for the fortifying herbal composition, by weight percentage: the bovine thymus is about 8.693%; the Astragalus roots are about 8.196%; the bovine red bone marrow is about 8.072%; the turmeric is about 7.451%; the bovine colostrum is about 5.588%; the bovine immunoglobulin and vitamin C are each about 4.967%; the turkey tail mushrooms are about 3.726%; the Eleuthera roots and Sambucus nigra are each about 3.105%; the cats claw bark, lactoferrin, licorice, and the olive leaf are each about 2.484%; the Adhatoda vasica, ashwagandha, bee glue, reishi mushrooms, and shiitake mushrooms are each about 2.235%; the rose hip is about 1.987%; the arabinogalactan is about 1.739%; the beta nicotinamide mononucleotide, bovine serum albumin, Echinacea pallida roots, Echinacea purpurea, red marine algae, rosemary leaf, and Thuja occidentals leaf are each about 1.242%; the beta-glucan and transfer factor are each about 0.993%; the umcka, vitamin E, and wild indigo roots are each about 0.745%; the oregano and zinc are each about 0.621%; the lysozyme is about 0.497%; the bee pollen is about 0.373%; the melatonin is about 0.298%; the bentonite clay and royal jelly are each about 0.248%; the adenosine triphosphate, LH01-Myoviridae, LL12-Myoviridae, LL15-Siphoviridae, and T4D-Myroviridae are each about 0.124%; the lycopene is about 0.099%; the vitamin D3 is about 0.072%; the vitamin A is about 0.025%; the selenium is about 0.005%; and the vitamin B12 is about 0.001%.

In some embodiments, the fortifying herbal composition further comprises Triphala, Andrographis paniculata, beleric myrobalan, chebulic myrobalan, papaya fruit, and Thymic Protein A. In further embodiments for the fortifying herbal composition, by weight percentage the Triphala, bovine thymus, Astragalus roots, bovine red bone marrow, and turmeric are each about 5% to about 20%; the Andrographis paniculata, bovine colostrum, bovine immunoglobulin, vitamin C, turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%; the beta nicotinamide mononucleotide, bovine serum albumin, Echinacea pallida roots, Echinacea purpurea, papaya fruit, red marine algae, rosemary leaf, and Thuja occidentals leaf are each about 0.5 to about 5%; the beta-glucan, transfer factor, umcka, vitamin E, wild indigo roots, oregano, zinc, lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 2%; the lycopene, vitamin D3, vitamin A, selenium, and vitamin B12 are each about 0.0005% to about 0.3%; and the Thymic Protein A is about 0.0001% to about 0.3%.

In additional embodiments for the fortifying herbal composition, by weight percentage: the Triphala is about 5% to about 15%; the bovine thymus, Astragalus roots, bovine red bone marrow, and turmeric are each about 5% to about 10%; the Andrographis paniculata, bovine colostrum, bovine immunoglobulin, and vitamin C are each about 3% to about 8%; the turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%; the beta nicotinamide mononucleotide, bovine serum albumin, Echinacea pallida roots, Echinacea purpurea, papaya fruit, red marine algae, rosemary leaf, and Thuja occidentals leaf are each about 1% to about 3%; the beta-glucan, transfer factor, umcka, vitamin E, wild indigo roots, oregano, and zinc are each about 0.3% to about 1.5%; the lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 1%; the lycopene, vitamin D3, vitamin A, selenium, and vitamin B12 are each about 0.0005% to about 0.1%; and the Thymic Protein A is about 0.0001% to about 0.1%.

In still further embodiments for the fortifying herbal composition, by weight percentage: the Triphala is about 9.9%; the bovine thymus is about 7.0%; the Astragalus roots is about 6.6%; the bovine red bone marrow is about 6.5%; the turmeric is about 6.0%; the Andrographis panticulata is about 5.0%; the bovine colostrum is about 4.5%; the bovine immunoglobulin and vitamin C are each about 4.0%; the turkey tail mushrooms are about 3.0%; the Eleuthera roots and Sambucus nigra are each about 2.5%; the beleric myrobalan, chebulic myrobalan, cats claw bark, lactoferrin, licorice, and olive leaf are each about 2.0%; the Adhatoda vasica, ashwagandha, bee glue, reishi mushrooms, and shiitake mushrooms are each about 1.8%; the rose hip is about 1.6%; the arabinogalactan is about 1.4%; the beta nicotinamide mononucleotide, bovine serum albumin, Echinacea pallida roots, Echinacea purpurea, papaya fruit, red marine algae, rosemary leaf, and Thuja occidentals leaf are each about 1.0%; the beta-glucan and transfer factor are each about 0.8%; the umcka, vitamin E, and wild indigo root are each about 0.6%; the oregano and zinc are each about 0.5%; the lysozyme is about 0.4%; the bee pollen is about 0.3%; the melatonin is about 0.24%; the bentonite clay and royal jelly are each about 0.2%; the adenosine triphosphate, LH01-myoviridae, LL12-myroviridae, LL15-Siphoviridae, and T4D-myroviridae are each about 0.1%; the lycopene is about 0.08%; the vitamin D3 is about 0.058%; the vitamin A is about 0.022%; the selenium is about 0.004%; the vitamin B12 is about 0.001%; and the Thymic Protein A is about 0.00024%.

Another aspect of the disclosure herein includes for, in some embodiments, a method for bolstering the human immune system of a patient against infections comprising: providing a dose of a fortifying composition at least once daily for at least one day; wherein the fortifying composition comprises bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

In some embodiments of the method, of the fortifying composition, by weight percentage: the bovine thymus is about 8.693%; the Astragalus roots are about 8.196%; the bovine red bone marrow is about 8.072%; the turmeric is about 7.451%; the bovine colostrum is about 5.588%; the bovine immunoglobulin and vitamin C are each about 4.967%; the turkey tail mushrooms are about 3.726%; the Eleuthera roots and Sambucus nigra are each about 3.105%; the cats claw bark, lactoferrin, licorice, and the olive leaf are each about 2.484%; the Adhatoda vasica, ashwagandha, bee glue, reishi mushrooms, and shiitake mushrooms are each about 2.235%; the rose hip is about 1.987%; the arabinogalactan is about 1.739%; the beta nicotinamide mononucleotide, bovine serum albumin, Echinacea pallida roots, Echinacea purpurea, red marine algae, rosemary leaf, and Thuja occidentals leaf are each about 1.242%; the beta-glucan and transfer factor are each about 0.993%; the umcka, vitamin E, and wild indigo roots are each about 0.745%; the oregano and zinc are each about 0.621%; the lysozyme is about 0.497%; the bee pollen is about 0.373%; the melatonin is about 0.298%; the bentonite clay and royal jelly are each about 0.248%; the adenosine triphosphate, LH01-Myoviridae, LL12-Myoviridae, LL15-Siphoviridae, and T4D-Myroviridae are each about 0.124%; the lycopene is about 0.099%; the vitamin D3 is about 0.072%; the vitamin A is about 0.025%; the selenium is about 0.005%; and the vitamin B12 is about 0.001%.

In other embodiments of the method, the fortifying composition further comprises comprising Triphala, Andrographis paniculata, beleric myrobalan, chebulic myrobalan, papaya fruit, and Thymic Protein A. In further embodiments, of the fortifying composition, by weight percentage: the Triphala, bovine thymus, Astragalus roots, bovine red bone marrow, and turmeric are each about 5% to about 20%; the Andrographis paniculata, bovine colostrum, bovine immunoglobulin, vitamin C, turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%; the beta nicotinamide mononucleotide, bovine serum albumin, Echinacea pallida roots, Echinacea purpurea, papaya fruit, red marine algae, rosemary leaf, and Thuja occidentals leaf are each about 0.5 to about 5%; the beta-glucan, transfer factor, umcka, vitamin E, wild indigo roots, oregano, zinc, lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 2%; the lycopene, vitamin D3, vitamin A, selenium, and vitamin B12 are each about 0.0005% to about 0.3%; and the Thymic Protein A is about 0.0001% to about 0.3%.

In still further embodiments of the method, of the fortifying composition, by weight percentage: the Triphala is about 5% to about 15%; the bovine thymus, Astragalus roots, bovine red bone marrow, and turmeric are each about 5% to about 10%; the Andrographis paniculata, bovine colostrum, bovine immunoglobulin, and vitamin C are each about 3% to about 8%; the turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%; the beta nicotinamide mononucleotide, bovine serum albumin, Echinacea pallida roots, Echinacea purpurea, papaya fruit, red marine algae, rosemary leaf, and Thuja occidentals leaf are each about 1% to about 3%; the beta-glucan, transfer factor, umcka, vitamin E, wild indigo roots, oregano, and zinc are each about 0.3% to about 1.5%; the lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 1%; the lycopene, vitamin D3, vitamin A, selenium, and vitamin B12 are each about 0.0005% to about 0.1%; and the Thymic Protein A is about 0.0001% to about 0.1%.

In still further embodiments of the method, of the fortifying composition, by weight percentage: the Triphala is about 9.9%; the bovine thymus is about 7.0%; the Astragalus roots is about 6.6%; the bovine red bone marrow is about 6.5%; the turmeric is about 6.0%; the Andrographis panticulata is about 5.0%; the bovine colostrum is about 4.5%; the bovine immunoglobulin and vitamin C are each about 4.0%; the turkey tail mushrooms are about 3.0%; the Eleuthera roots and Sambucus nigra are each about 2.5%; the beleric myrobalan, chebulic myrobalan, cats claw bark, lactoferrin, licorice, and olive leaf are each about 2.0%; the Adhatoda vasica, ashwagandha, bee glue, reishi mushrooms, and shiitake mushrooms are each about 1.8%; the rose hip is about 1.6%; the arabinogalactan is about 1.4%; the beta nicotinamide mononucleotide, bovine serum albumin, Echinacea pallida roots, Echinacea purpurea, papaya fruit, red marine algae, rosemary leaf, and Thuja occidentals leaf are each about 1.0%; the beta-glucan and transfer factor are each about 0.8%; the umcka, vitamin E, and wild indigo root are each about 0.6%; the oregano and zinc are each about 0.5%; the lysozyme is about 0.4%; the bee pollen is about 0.3%; the melatonin is about 0.24%; the bentonite clay and royal jelly are each about 0.2%; the adenosine triphosphate, LH01-myoviridae, LL12-myroviridae, LL15-Siphoviridae, and T4D-myroviridae are each about 0.1%; the lycopene is about 0.08%; the vitamin D3 is about 0.058%; the vitamin A is about 0.022%; the selenium is about 0.004%; the vitamin B12 is about 0.001%; and the Thymic Protein A is about 0.00024%.

In some embodiments, the dose is about 1000 mg, and the dose is provided four times daily. In other embodiments, the dose is about 1250 mg, and the dose is provided four times daily. In some embodiments, the dose is provided at least once a day for at least fourteen consecutive days.

In some embodiments, after providing the dose of the fortifying composition, at least one of the markers comprising: CD8a, CD4, CD11b, TCR-alpha, or Ly6G is present at an increased level in the patient following exposure to an infection. In other embodiments, at least two of the markers comprising: CD8a, CD4, CD11b, TCR-alpha, or Ly6G are present at an increased level in the patient following exposure to an infection. In further embodiments, all of the markers comprising: CD8a, CD4, CD11b, TCR-alpha, or Ly6G are present at an increased level in the patient following exposure to an infection.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 depicts one embodiment of a first herbal mixture by weight percentage for bolstering human immune response to viral infections.

FIG. 2 depicts one embodiment a second herbal mixture by weight percentage for bolstering human immune response to viral infections.

FIG. 3 depicts one embodiment of a third herbal mixture by weight percentage for fortifying human immune response to viral infections.

FIG. 4 depicts another embodiment of a third herbal mixture by weight percentage for fortifying human immune response to viral infections.

FIG. 5 illustrates one embodiment of a method for treating or preventing viral infections and bolstering human immune response to viral infections.

FIGS. 6-11 depict flow cytometry results from blood samples of an in vivo experimental study in Sprague Dawley rats.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

With the extent of the 2020 COVID-19 global health crisis, there is an overwhelming need for new compositions and methods for improving human health, in particular to bolstering the human immune response to viral infections. Discussed herein are various embodiments of herbal mixtures and therapeutic methods that can be employed by a patient or a medical practitioner separately or in any combination, including all embodiments together. Each of these herbal mixtures and methods will be described below separately, and in some combinations, below in order to enable one of skill in the art to make and use the contemplated invention(s). The described combinations should not be understood to limit or exclude any other combinations possible among the various features.

As used herein, the terms “herbal composition” and “herbal mixture” can be used interchangeably as will be appreciated by one of skill in the art. In any given example, either term will be chosen where more appropriate for the purposes of clarity and readability.

As described herein, the various herbal compositions can be formulated in order to be administered orally, parenterally, topically, or rectally as a suppository. For the purposes of describing the herbal mixtures, it is easiest to discuss them as a dry powder in many embodiments. However, in other embodiments, the herbal mixtures are first used to produce an infusion in hot water or another liquid that is then drunk or further formulated. In other embodiments, the dry powders are packaged in pharmaceutically acceptable capsules and taken orally. The description herein of any herbal composition is intended to cover all formulations that necessitate, imply, or are equivalent to the described ingredients in their described quantities as dry powders, regardless of any further mechanical processing or formulating.

As used herein, many of the ingredients of the herbal mixtures are dry powders in many embodiments. One of skill in the art will appreciate that many of these ingredients are commercially available as dry powders. If the ingredients are unavailable in this form, the ingredients must be dried (e.g., by practices such as sun drying, drum drying, convection drying, with a conventional oven and/or an electric food dehydrator, etc.) and subsequently ground (e.g., with a mortar and pestle, a food processor, etc.). One of skill in the art will also appreciate that many of the ingredients are available as extracts, which can involve steeping, dissolving, or further processing the full ingredient in a solvent, the resulting liquid of which is subsequently evaporated to produce a dry powder of “extract.” For the purposes of clarity and readability, the term “extract” is avoided in the recitation of the ingredients but indicated the below list of ingredients where appropriate.

As used herein, COVID-19 refers to the Severe Acute Respiratory Syndrome Coronavirus 2 (SARS-CoV-2) that made global news in late 2019 with worldwide infections.

As used herein, “anti-inflammatory” indicates that the herbal ingredient, or mixture as a whole, is capable of interfering or interacting with the human body's inflammatory or inflammation inducing pathways in a manner to reduce the symptoms of inflammation (e.g., swelling, discoloration, pain, etc.). In many embodiments, these anti-inflammatory effects are achieved by interfering or interacting with pro-inflammatory cytokines (e.g., Interleukin-6, which has special relevance to the COVID-19 epidemic) and their related signaling pathways; however, the term “anti-inflammatory” as used herein is also intended to cover other means and interactions with other biological systems and pathways that yield similar reductions in the symptoms of inflammation.

As used herein, “a booster of the immune system” and similar phrases indicate that the ingredient, the herbal mixture as a whole, or the method can provide various benefits to a human subject's immune system in a way that facilitates its capacities in one or both of preventing or fighting an infection (e.g., viral, bacterial, or other). This “immune system boosting” can be achieved in a variety of ways, such as by stimulating white blood cell count or relevant signaling molecule activity or post translational modifications, increasing blood flow or transport or migration of relevant cells and molecules (e.g., chemotaxis) throughout the body, providing high quantities of antioxidant compounds in order to mitigate natural cell damage and thereby improving cells' resistance to infection, and more.

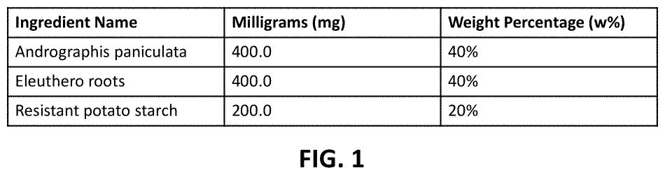

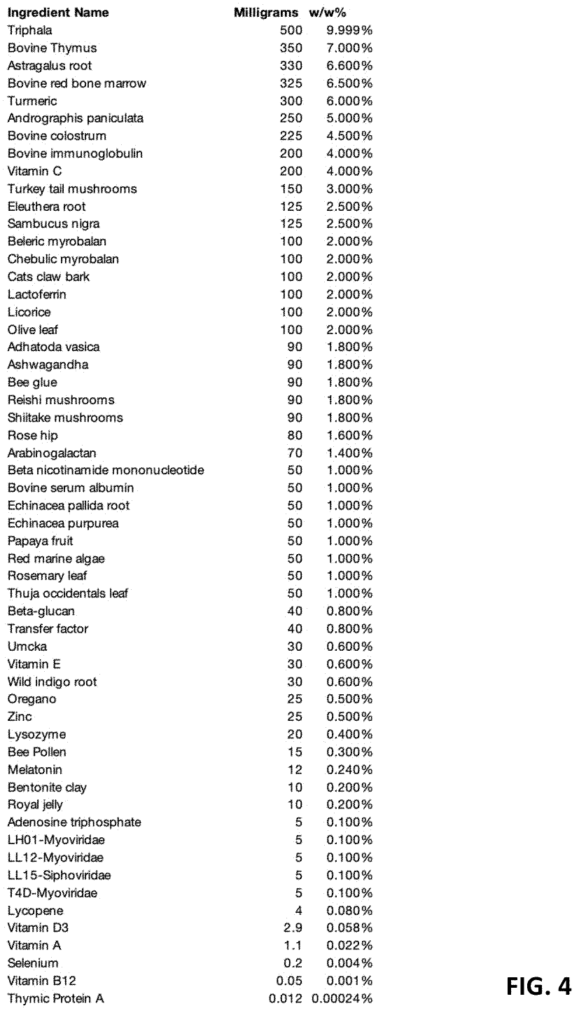

FIG. 1 depicts a table describing a first herbal composition for treating or preventing a viral infection in a human patient. In some embodiments, this first herbal composition can be considered a “pretreatment” herbal composition as it can be provided to a patient for a few days prior to the provision of subsequent treatment regimens as is described below in various embodiments in FIG. 4. Alternatively, the first herbal composition may be administered alone, without any additional treatment regimens. The term “pretreatment” is meant only to be a convenient identifier for the first herbal composition to distinguish it from any other herbal compositions discussed herein, and the term should not be interpreted to indicate any biological or physiological activity (or a lack thereof) beyond those of the properties of the herbal composition itself.

This first herbal composition comprises Andrographis paniculata, Eleuthero roots, and resistant potato starch. These ingredients will be described in further detail below. In some embodiments, these ingredients can be present in any proportion. In another embodiment, the first herbal composition comprises about 10% to about 90% Andrographis paniculata, about 10% to about 90% Eleuthero roots, and about 5% to about 50% resistant potato starch by weight percentage. In a further embodiment, the first herbal composition comprises about 20% to about 80% Andrographis paniculata, about 20% to about 80% Eleuthero roots, and about 10% to about 40% resistant potato starch by weight percentage. In still another embodiment, such as the embodiment of FIG. 1, the first herbal composition comprises about 40% Andrographis paniculata, about 40% Eleuthero roots, and about 20% resistant potato starch by weight percentage. For example, a composition with these proportions can be made by mixing about 400 mg of Andrographis paniculata with about 400 mg of Eleuthero roots and about 200 mg of resistant potato starch. Any of the above embodiments can additionally include, individually or in combination, sweeteners (natural and/or artificial), colorants (natural and/or artificial), surfactants, thickeners, and/or any other compound for the purposes of material processing, handling, and mixture palatability without deviating from the scope of this disclosure.

To manufacture the first herbal composition of FIG. 1, the ingredients must be dried or be acquired properly dried. As discussed above, one of skill in the art will appreciate the various means available to do this. Next, each ingredient must be individually ground into a fine powder or be acquired as a dry, fine powder. As discussed above, one of skill in the art will appreciate the various means available to do this. Once each ingredient has been turned into a powdered state, they are then combined in a single vessel or container and thoroughly mixed, either by hand or with any automatic tool, such as a food processor, blender, or industrial mixer, to evenly distribute each ingredient throughout the powdered mixture.

For the purposes of describing the first herbal mixture, it is easiest to discuss it as a dry powder. However, in some embodiments, the first herbal mixture is administered to a patient as an infusion in hot water that is then drunk. To produce this drink, a certain mass of the herbal mixture (i.e., a “dose”) is placed in a vessel containing a about 0.5 to 3 cups of hot or boiling water, and the mixture is allowed to steep in the hot water for at least about 5 minutes after which any remaining physical components are removed from the liquid, usually by straining. In some embodiments, the vessel is closed with a lid during the duration of the steeping. In one embodiment, about 1000 mg of the first herbal mixture are placed within about 1 to about 1.5 cups (i.e., approximately 300 mL) of boiling water and allowed to steep within a sealed vessel for about five to about fifteen minutes, for example about twelve minutes. After steeping, any remaining solid components are removed from the liquid by straining.

In alternative embodiments, the dry powder composition can be packaged into capsules and taken orally. In some embodiments, the capsules are composed of a vegan gelatin substance, but one of skill in the art will recognize that any pharmaceutically acceptable capsule material can be employed. In even further embodiments, as discussed above, the first herbal mixture, as a dry powder or as an infusion, can be further formulated into a variety of means for administration to a human patient.

In some embodiments, the immune-boosting and anti-inflammatory properties of the first herbal mixture strongly combat symptoms of various viral infections, including COVID-19. In other embodiments, the first herbal mixture is similarly able to combat symptoms of other respiratory infections, such as pneumonia. Furthermore, in certain embodiments, the first herbal mixture is able to combat the virus itself, disrupting its ability to enter cells and replicate. In still other embodiments, the first herbal mixture's anti-inflammatory properties come from, at least in part, to its ability to reduce the levels of Interleukin 6 (IL-6), a pro-inflammatory cytokine, in the human body.

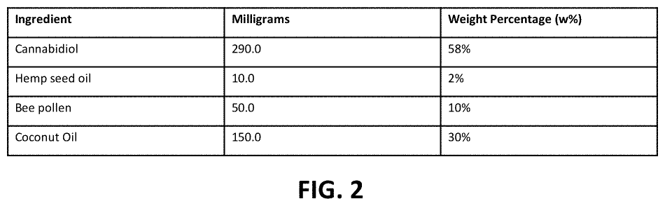

FIG. 2 depicts a table describing a second herbal composition for treating or preventing a viral infection in a human patient. In some embodiments, this second herbal composition can be considered a “treatment” herbal composition, as it can be provided to a patient on at least some of the same days as the provision of additional treatment regimens such as what is described below in various embodiments in FIG. 4. Alternatively, the second herbal composition may be administered alone, without any additional treatment regimens. The term “treatment” is meant only to be a convenient identifier for the second herbal composition to distinguish it from any other herbal compositions discussed herein, and the term should not be interpreted to indicate any biological or physiological activity (or a lack thereof) beyond those of the properties of the herbal composition itself.

The second herbal composition comprises cannabidiol, bee pollen, and coconut oil. In certain embodiments, the second herbal composition additionally comprises hemp seed oil. These ingredients are discussed in greater detail below. In some embodiments, these ingredients can be present in any proportions. In another embodiment, the second herbal mixture comprises about 30% to about 90% cannabidiol, about 0.5% to about 25% hemp seed oil, about 5% to about 30% bee pollen, and about 15% to about 60% coconut oil by weight percentage. In another embodiment, the second herbal composition comprises about 30% to about 90% cannabidiol, about 1% to about 10% hemp seed oil, about 5% to about 15% bee pollen, and about 15% to about 45% coconut oil by weight percentage. In still another embodiment, the embodiment of FIG. 2, the second herbal composition comprises about 58% cannabidiol, about 2% hemp seed oil, about 10% bee pollen, and about 30% coconut oil by weight percentage. For example, a composition with these proportions can be made by mixing about 290 mg of cannabidiol and 10 mg of hemp seed oil with about 50 mg of bee pollen and 150 mg of coconut oil. In an alternative embodiment without hemp seed oil, the treatment herbal composition comprises about 60% cannabidiol, about 10% bee pollen, and 30% coconut oil. Any of the above embodiments can additionally include individually or in combination sweeteners (natural and/or artificial), colorants (natural and/or artificial), surfactants, and thickeners, and/or any other compound for the purposes of material processing, handling and mixture palatability without deviating from the scope of this disclosure.

To manufacture the second herbal composition, bee pollen can be dried and ground into a powder as will be appreciated by those of skill in the art, or it can be acquired in this state. It can then be suspended and thoroughly mixed into the coconut oil by any number of means available to those of skill. Cannabidiol can be acquired as either a dry powder or as an oil. In embodiments where dry, powdered cannabidiol is used, the powder can be mixed into the coconut oil in a manner similar to the bee pollen. In embodiments where cannabidiol-containing oil is used, one of skill in the art will appreciate how to measure out enough of the oil in order to meet the weight proportions for the cannabidiol described herein for various embodiments by calculating out the weight fraction of the cannabidiol from the quantity of other compounds present in the oil. In many embodiments, doses of the second herbal composition are given orally by consuming the resulting oil mixture. In an alternative embodiment, the resulting mixture is packaged into a capsule and taken orally. In some embodiments, the capsules are made of a vegan gelatin substance, but one of skill in the art will recognize that any pharmaceutically acceptable capsule material can be employed. In other embodiments, as discussed above, the composition as an oil can be further formulated for various means of delivery.

In some embodiments, the anti-inflammatory and immune-boosting properties of the second herbal composition allow it to combat both symptoms of viral infections as well as the virus itself. Furthermore, the second herbal composition's ability to provide pain relief in certain embodiments, which can be useful when the second herbal composition is taken in parallel to other treatment regimens that can provide pain or discomfort (such as the method of FIG. 4 in certain embodiments as discussed below).

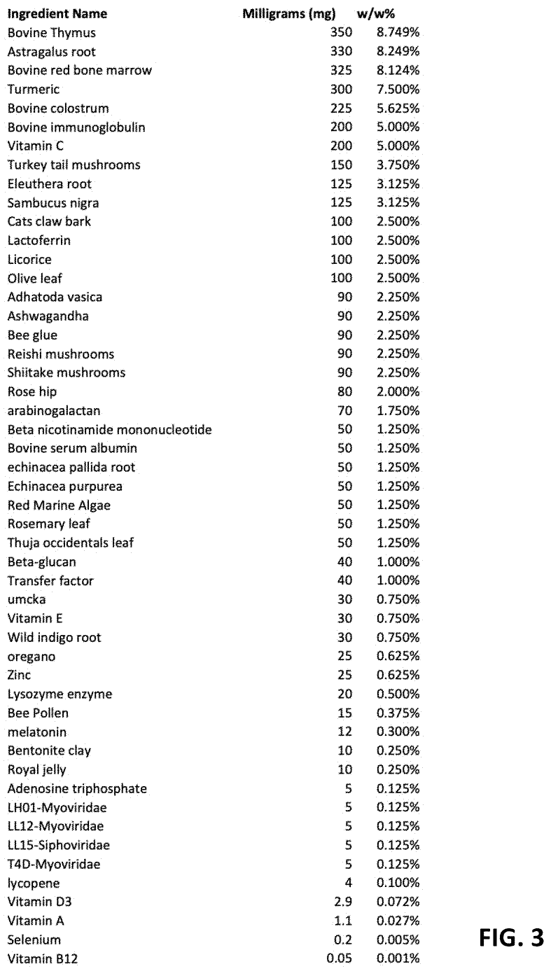

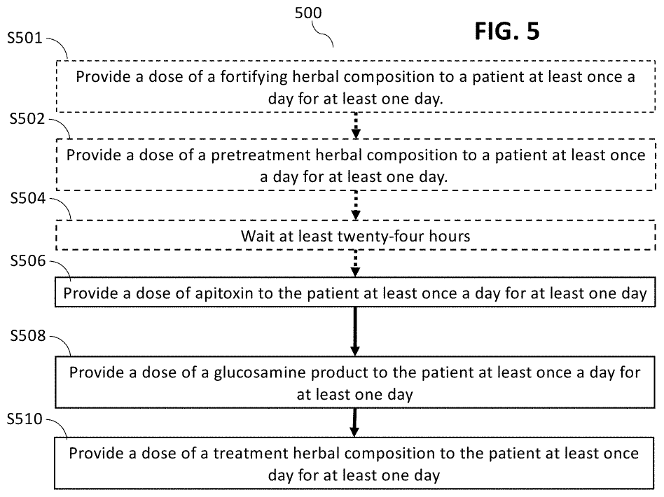

FIGS. 3 and 4 depict tables describing embodiments of a third herbal composition for treating or preventing a viral infection in a human patient. In some embodiments, this third herbal composition can be considered a “fortifying” herbal composition, as it can be provided to a patient before, during, or after the provision of additional treatment regimens such as what is described below in various embodiments in FIG. 5. Alternatively, the third herbal composition may be administered alone, without any additional treatment regimens. The term “fortifying” is meant only to be a convenient identifier for the third herbal composition to distinguish it from any other herbal compositions discussed herein, and the term should not be interpreted to indicate any biological or physiological activity (or a lack thereof) beyond those of the properties of the herbal composition itself.

In many embodiments, the third herbal composition comprises bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12. These ingredients are discussed in greater detail below. In a further embodiment, the fortifying composition additionally comprises Triphala, Andrographis paniculata, beleric myrobalan, chebulic myrobalan, papaya fruit, and Thymic Protein A.

The ingredients of the third herbal composition can be present in different proportions according to various embodiments. In one embodiment, described as a weight percentage, the bovine thymus, Astragalus roots, bovine red bone marrow, and turmeric are each about 5% to about 20%, the bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%, the beta nicotinamide mononucleotide, bovine serum albumin, Echinacea pallida roots, Echinacea purpurea, red marine algae, rosemary leaf, and Thuja occidentals leaf are each about 0.5 to about 5%, the beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 2%, and the lycopene, vitamin D3, vitamin A, selenium, and vitamin B12 are each about 0.0005% to about 0.3%. In another embodiment, also described as a weight percentage, the bovine thymus, Astragalus roots, bovine red bone marrow, and turmeric are each about 5% to about 10%, the bovine colostrum, bovine immunoglobulin, and vitamin C are each about 3% to about 8%, the turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%, the beta nicotinamide mononucleotide, bovine serum albumin, Echinacea pallida roots, Echinacea purpurea, red marine algae, rosemary leaf, and Thuja occidentals leaf are each about 1% to about 3%, the beta-glucan, transfer factor, umcka, vitamin E, wild indigo roots, oregano, and zinc are each about 0.3% to about 1.5%, the lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 1%, and the lycopene, vitamin D3, vitamin A, selenium, and vitamin B12 are each about 0.0005% to about 0.1%.

In still another embodiment, the specific embodiment depicted in FIG. 3, described by weight percentage, the bovine thymus is about 8.693%, the Astragalus roots are about 8.196%, the bovine red bone marrow is about 8.072%, the turmeric is about 7.451%, the bovine colostrum is about 5.588%, the bovine immunoglobulin and vitamin C are each about 4.967%, the turkey tail mushrooms are about 3.726%, the Eleuthera roots and Sambucus nigra are each about 3.105%, the cats claw bark, lactoferrin, licorice, and the olive leaf are each about 2.484%, the Adhatoda vasica, ashwagandha, bee glue, reishi mushrooms, and shiitake mushrooms are each about 2.235%, the rose hip is about 1.987%, the arabinogalactan is about 1.739%, the beta nicotinamide mononucleotide, bovine serum albumin, Echinacea pallida roots, Echinacea purpurea, red marine algae, rosemary leaf, and Thuja occidentals leaf are each about 1.242%, the beta-glucan and transfer factor are each about 0.993%, the umcka, vitamin E, and wild indigo roots are each about 0.745%, the oregano and zinc are each about 0.621%, the lysozyme is about 0.497%, the bee pollen is about 0.373%, the melatonin is about 0.298%, the bentonite clay and royal jelly are each about 0.248%, the adenosine triphosphate, LH01-Myoviridae, LL12-Myoviridae, LL15-Siphoviridae, and T4D-Myroviridae are each about 0.124%, the lycopene is about 0.099%, the vitamin D3 is about 0.072%, the vitamin A is about 0.025%, the selenium is about 0.005%, and the vitamin B12 is about 0.001%. An example of such an embodiment can be made by mixing the milligram (mg) quantities listed in the table of FIG. 3 in order to generate 4000.25 mg mixture that has these proportions. Any of the above embodiments can additionally include individually, or in combination, sweeteners (natural and/or artificial), colorants (natural and/or artificial), surfactants, and thickeners, and/or any other compound for the purposes of material processing, handling, and mixture palatability without deviating from the scope of this disclosure.

In one embodiment, described as a weight percentage, the Triphala, bovine thymus, Astragalus roots, bovine red bone marrow, and turmeric are each about 5% to about 20%, the Andrographis paniculata, bovine colostrum, bovine immunoglobulin, vitamin C, turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%, the beta nicotinamide mononucleotide, bovine serum albumin, Echinacea pallida roots, Echinacea purpurea, papaya fruit, red marine algae, rosemary leaf, and Thuja occidentals leaf are each about 0.5 to about 5%, the beta-glucan, transfer factor, umcka, vitamin E, wild indigo roots, oregano, zinc, lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 2%, the lycopene, vitamin D3, vitamin A, selenium, and vitamin B12 are each about 0.0005% to about 0.3%, and the Thymic Protein A is about 0.0001% to about 0.3%. In another embodiment, also described as a weight percentage, the Triphala is about 5% to about 15%, the bovine thymus, Astragalus roots, bovine red bone marrow, and turmeric are each about 5% to about 10%, the Andrographis paniculata, bovine colostrum, bovine immunoglobulin, and vitamin C are each about 3% to about 8%, the turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%, the beta nicotinamide mononucleotide, bovine serum albumin, Echinacea pallida roots, Echinacea purpurea, papaya fruit, red marine algae, rosemary leaf, and Thuja occidentals leaf are each about 1% to about 3%, the beta-glucan, transfer factor, umcka, vitamin E, wild indigo roots, oregano, and zinc are each about 0.3% to about 1.5%, the lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 1%, the lycopene, vitamin D3, vitamin A, selenium, and vitamin B12 are each about 0.0005% to about 0.1%, and the Thymic Protein A is about 0.0001% to about 0.1%.

In the specific embodiment depicted in FIG. 4, described by weight percentage, the Triphala is about 9.9%, the bovine thymus is about 7.0%, the Astragalus roots is about 6.6%, the bovine red bone marrow is about 6.5%, the turmeric is about 6.0%, the Andrographis panticulata is about 5.0%, the bovine colostrum is about 4.5%, the bovine immunoglobulin and vitamin C are each about 4.0%, the turkey tail mushrooms are about 3.0%, the Eleuthera roots and Sambucus nigra are each about 2.5%, the beleric myrobalan, chebulic myrobalan, cats claw bark, lactoferrin, licorice, and olive leaf are each about 2.0%, the Adhatoda vasica, ashwagandha, bee glue, reishi mushrooms, and shiitake mushrooms are each about 1.8%, the rose hip is about 1.6%, the arabinogalactan is about 1.4%, the beta nicotinamide mononucleotide, bovine serum albumin, Echinacea pallida roots, Echinacea purpurea, papaya fruit, red marine algae, rosemary leaf, and Thuja occidentals leaf are each about 1.0%, the beta-glucan and transfer factor are each about 0.8%, the umcka, vitamin E, and wild indigo root are each about 0.6%, the oregano and zinc are each about 0.5%, the lysozyme is about 0.4%, the bee pollen is about 0.3%, the melatonin is about 0.24%, the bentonite clay and royal jelly are each about 0.2%, the adenosine triphosphate, LH01-myoviridae, LL12-myroviridae, LL15-Siphoviridae, and T4D-myroviridae are each about 0.1%, the lycopene is about 0.08%, the vitamin D3 is about 0.058%, the vitamin A is about 0.022%, the selenium is about 0.004%, the vitamin B12 is about 0.001%, and the Thymic Protein A is about 0.00024%. An example of such an embodiment can be made by mixing the milligram (mg) quantities listed in the table of FIG. 4 in order to generate 5000.262 mg mixture that has these proportions. Any of the above embodiments can additionally include individually, or in combination, sweeteners (natural and/or artificial), colorants (natural and/or artificial), surfactants, and thickeners, and/or any other compound for the purposes of material processing, handling, and mixture palatability without deviating from the scope of this disclosure.

To manufacture the third herbal composition of FIGS. 3 and 4, the ingredients must be dried or be acquired properly dried. As discussed above, one of skill in the art will appreciate the various means available to do this. Next, each ingredient must be individually ground into a fine powder or be acquired as a dry, fine powder. As discussed above, one of skill in the art will appreciate the various means available to do this. Once each ingredient has been turned into a powdered state, they are then combined in a single vessel or container and thoroughly mixed, either by hand or with any automatic tool, such as a food processor, blender, or industrial mixer, to evenly distribute each ingredient throughout the powdered mixture. In some embodiments, the dry powder composition can be packaged into capsules and taken orally. In some embodiments, the capsules are composed of a vegan gelatin substance, but one of skill in the art will recognize that any pharmaceutically acceptable capsule material can be employed. In even further embodiments, as discussed above, the first herbal mixture, as a dry powder or as an infusion, can be further formulated into a variety of means for administration to a human patient.

The fortifying herbal composition provides a wealth of ingredients that synergize to substantially boost the immune system. In some embodiments, this can include promoting DNA repair, increasing number and activity of white blood cells, including T-cells, B-cells, and natural killer cells, providing and fostering antibody distribution, and fostering healthy metabolism. In particular, the fortifying herbal composition can, in some embodiments, foster the growth and health of greater numbers of naïve T-cells, which can survive in the body for upwards of nine years, greatly outliving memory T-cells. Therefore, in some embodiments, promoting a higher population of healthy naïve T-cells can provide a substantial benefit to human health, as naïve T-cells are critical for the immune response to new pathogens.

### List of Ingredients

Adenosine Triphosphate—(ATP) the common biomolecule responsible for providing stored chemical energy to the body that drives many cellular processes. In some embodiments, as an ingredient, ATP provides further energy to the patient.

Adhatoda vasica—also known as Justicia adhatoda, Malabar nut, adulsa and adhatoda provides antioxidant, anti-inflammatory, and anti-microbial effects in some embodiments. In some embodiments, it is considered a booster of the immune system.

Andrographis paniculata—the plant also known as creat or green chireta. In some embodiments, an extract of Andrographis paniculata is used. In some embodiments, the extract provides anti-inflammatory and anti-viral activity.

Arabinogalactan—a naturally-occurring polysaccharide biopolymer of arabinose and galactose. In some embodiments, plant arabinogalactan is used. In further embodiments, the arabinogalactan is extracted from the larch tree (genus Larix). In some embodiments, it is considered a booster of the immune system by improving immune response. In some embodiments, it provides anti-cancer effects.

Ashwagandha—(Withania somnifera) also known as Indian ginseng, poison gooseberry, and winter cherry provides antioxidant effects in some embodiments. In further embodiments, it is considered a booster of the immune system. In some embodiments, powdered roots of the plant is used.

Astragalus root—roots of any plant of the genus Astragalus, which includes plants of the common names milkvetch, locoweed, and goat's-thorn. In some embodiments, an extract of Astragalus roots is used. In some embodiments, it is used to treat viral infections and to boost the immune system.

Bee Glue—(Propolis) a mixture of beeswax, bee saliva, and various plant exudates produced by honeybees (genus Apis) to seal gaps and provide structural stability to a hive. The exact composition of bee glue can vary regionally and seasonally. In some embodiments, it provides anti-viral, anti-microbial, and anti-inflammatory properties in addition to boosting the immune system.

Bee pollen—amalgamations of flower pollen collected by many species of honeybees (genus Apis). Bee pollen contains a variety of sugars, proteins, minerals, and other biomolecules having somewhat of a variance that is dependent upon the source flowers. In some embodiments, bee pollen provides anti-microbial and immune-boosting effects when consumed.

Beleric myrobalan—the fruit of the tree (Terminala bellirica) also known as Bibhitaki, baheda and bahera. In some embodiments, the extract of the fruit with or without the seeds can be used. In some embodiments, it is considered as a booster of the immune system.

Bentonite Clay—an aluminum phyllosilicate mineral that can contain a wealth of trace minerals. In some embodiments, it provides antibacterial effects in addition to general infection-fighting and immune system boosting capacities.

Beta nicotinamide mononucleotide—(NMN) a nucleotide comprising a ribose sugar and nicotinamide. In some embodiments, NMN provides anti-aging effects and can improve the repair of DNA.

Beta-glucan—polysaccharide chains of Beta-D-glucose molecules. In some embodiments, an extract of beta-glucans is used. In some embodiments, beta-glucan extracts can boost the immune system, namely by improving the immune response of large effector cells. In some embodiments, beta 1,3-1,6 glucans are used. In further embodiments, these beta 1,3-1,6 glucans are derived from yeast (S. cerevisiae.)

Bovine colostrum—also known as beestings, bisnings, and first milk. It is the milk from the cow (genus Bos) produced soon after giving birth. It differs from normal milk in at least that it contains various antibodies. In some embodiments, it provides high concentrations of the antibodies IgA and IgM, thereby boosting the immune system with anti-viral and antibacterial effects.

Bovine immunoglobulin—immunoglobulin collected from the cow (genus Bos). In some embodiments, the immunoglobulin is at least at least 52% IgG. By providing additional antibodies, it is considered a booster of the immune system in some embodiments.

Bovine red bone marrow—red bone marrow collected from the cow (genus Bos). It can contain the hormone adiponectin that can assist in digestion and insulin regulation. In some embodiments, it is considered a booster of the immune system by providing myeloid and lymphoid stem cells.

Bovine serum albumin—albumin collected from the blood serum of the cow (genus Bos). In some embodiments, it assists stabilizing certain enzymes during digestion.

Bovine thymus—powdered thymus of the cow (genus Bos). In some embodiments, the ingredient is first lyophilized. In some embodiments, it is considered a booster of the immune system. In further embodiments, it can increase the count of white blood cells and T helper cells.

Cannabidiol (CBD)—a phytocannabidoid extracted from the cannabis plant. In some embodiments, it provides pain relief and anti-inflammatory activity.

Cats claw bark—bark of the woody vine Uncaria tomentosa. In some embodiments it provides antiviral and anticancer effects in addition to boosting the immune system.

Chebulic myrobalan—fruit of the tree also known as black myrobalan (Terminalia chebula) or Haritaki. In some embodiments, the extract of the fruit with or without the seeds can be used. In some embodiments, it contributes to healthy liver and kidney functions.

Coconut Oil—also called copra oil, is the oil collected from the coconuts of the coconut palm (Cocos nucifera). In some embodiments, the edible oil provides a flavorful medium for other components in addition to being a strong source of high-caloric saturated fats.

Echinacea pallida root—the root of the flower commonly known as the pale purple coneflower. In some embodiments, an extract of Echinacea pallida root is used. In some embodiments, it provides benefits against infections including viral infections and can be considered a booster of the immune system.

Echinacea purpurea—also known as the eastern purple coneflower, purple coneflower, hedgehog cornflower, and echinacea. It some embodiments it provides benefits against infections including viral infections and can be considered a booster of the immune system.

Eleuthero roots—(Eleutherococcus senticosus) also called Siberian ginseng and devil's bush. In some embodiments, an extract from Eleuthero roots is used. In some embodiments, it provides anti-inflammatory and anti-viral properties.

Hemp seed oil—oil produced from the pressing of the seeds of the hemp plant (a strain of Cannabis sativa). Edible, it primarily contains polyunsaturated fats including healthy omega-6 and omega-3 fatty acids. For at least this reason, it can be used as a dietary supplement.

Lactoferrin—also known as lactotransferrin, a glycoprotein commonly found in human colostrum and breast milk. In some embodiments, its apo form (i.e., apolactoferrin) is used. As used herein, proportional weights are calculated according to its apolactoferrin form. In some embodiments, it provides antiviral effects. In further embodiments, it provides immune boosting effects.

LH01-Myoviridae—a bacteriophage. In some embodiments, it provides antibacterial effects. In other embodiments, it can stimulate the production of antibodies and other mechanisms of the human immune system. In some embodiments, it is considered a booster of the immune system. (See also LL12-Myoviridae, LL15-Siphoviridae, and T4D-Myoviridae).

Licorice—the plant known as Glycyrrhiza glabra commonly used in candies and confectionaries. In some embodiments, it provides antiviral effects.

LL12-Myoviridae—a bacteriophage. In some embodiments, it provides antibacterial effects. In other embodiments, it can stimulate the production of antibodies and other mechanisms of the human immune system. In some embodiments, it is considered a booster of the immune system. (See also LH01-Myoviridae, LL15-Siphoviridae, and T4D-Myoviridae).

LL15-Siphoviridae—a bacteriophage. In some embodiments, it provides antibacterial effects. In other embodiments, it can stimulate the production of antibodies and other mechanisms of the human immune system. In some embodiments, it is considered a booster of the immune system. (See also LH01-Myoviridae, LL12-Myoviridae, and T4D-Myoviridae).

Lycopene—a carotenoid found in many fruits and vegetables, including tomatoes. In some embodiments, it protects the body from oxidative damage and can be considered a booster of the immune system.

Lysozyme—also known as muramidase and N-acetylmuramide glycanhydrolase, an enzyme found in the human body. In some embodiments, it provides antibacterial effects with its capability to damage certain bacteria cell walls. In other embodiments, it can also provide antiviral effects, inhibiting viral replication.

Melatonin—a hormone that plays a critical role in the regulation of sleep. In some embodiments, melatonin provides antiviral effects. In some embodiments, it can do this by blocking certain structures on the surface of viruses, including COVID-19. In certain embodiments, it is considered a booster of the immune system.

Olive leaf—leaves from the tree Olea europaea. In some embodiments, an extract of olive leaf is used. In some embodiments, it can provide antiviral and anti-inflammatory effects. In some embodiments, it is considered a booster of the immune system.

Oregano—the plant Origanum vulgare, sometimes known as wild marjoram. In some embodiments, an extract of oregano is used. In some embodiments, it provides antimicrobial and anti-toxin effects. It can be considered a booster of the immune system in some embodiments.

Papaya fruit—fruit of the plants of the genus Carica. In many embodiments, the fruit of the plant C. papaya (synonymous with C. peltata and C. posoposa) is used. In further embodiments, the flesh of fruit can be used in powdered form. In some embodiments, it provides anti-infectious activity and can be considered a booster of the immune system.

Red Marine Algae—powdered red marine algae. In some embodiments, the red marine algae Gigartina skottsbergii is used, though other red marine algae can be used in other embodiments. In some embodiments, an extract of red marine algae is used. In some embodiments, it is considered a booster of the immune system for provided broad spectrum benefits to it.

Reishi mushrooms—the fungus Ganoderma lingzhi also known as Lingzhi mushrooms. In some embodiments, it provides antiviral effects. In further embodiments, it is considered a booster of the immune system and can enhance white blood cell function. In some embodiments, it provides anti-cancer effects.

Resistant potato starch—the starch of potato plants (Solanum tuberosum) that is resistant to digestion in the human intestines. Instead digested by various gut bacteria, in some embodiments, it provides anti-inflammatory properties.

Rose hip—the fruit of the rose plant (genus Rosa). In some embodiments, an extract of rose hip is used. In some embodiments, it is considered a booster of the immune system for its broad spectrum benefits to it.

Rosemary leaf—leaves of the plant Salvia rosmarinus (synonymous with Rosmarinus officinalis) sometimes called anthos. In some embodiments, an extract of rosemary leaf is used. In some embodiments, it provides anti-inflammatory effects and can provide a high quantity of antioxidants.

Royal Jelly—the secretions of honeybees (genus Apis) used to feed developing honeybee larvae and queen bees and contains a mixture of biomolecules. In some embodiments, powdered royal jelly is used. In some embodiments, it provides anti-inflammatory effects. In other embodiments, it is considered a booster of the immune system and strengthens the immune response against bacterial and viral infections.

Sambucus nigra—the plant commonly known as elder, elderberry, black elderberry, and European black elderberry. In some embodiments, it provides antiviral and anti-inflammatory effects. It is considered a booster of the immune system in some embodiments.

Selenium—a chemical element and micronutrient. In some embodiments, L-selenomethionine, a naturally occurring amino acid containing selenium, is used as the source of selenium. In some embodiments, the L-selenomethionine is derived from black mustard seeds (Brassica nigra), but in other embodiments, other sources can be used. As used herein, proportional weights for “selenium” are calculated from L-selenomethionine. In some embodiments, it provides anti-inflammatory effects. In other embodiments, it is considered a booster of the immune system for its broad spectrum enhancements to that system.

Shiitake mushrooms—the fungus Lentinula edodes. In some embodiments, it provides anti-inflammatory effects. In other embodiments, it is considered a booster of the immune system for its broad spectrum enhancements to that system.

Triphala—a combination of the fruit of the three trees Amalaki (Phyllanthus emblica), Bibhitaki (Terminalia bellirica), and Haritaki (Terminalia chebula). In some embodiments, the extract of the fruits in equal proportions with or without their seeds can be used. In some embodiments, it is considered a booster of the immune system.

T4D-Myoviridae—a bacteriophage. In some embodiments, it provides antibacterial effects. In other embodiments, it can stimulate the production of antibodies and other mechanisms of the human immune system. In some embodiments, it is considered a booster of the immune system. (See also LH01-Myoviridae, LL12-Myoviridae, and LL15-Siphoviridae).

Thuja occidentals leaf—leaves of the tree commonly known as northern white-cedar, eastern white cedar, and eastern arborvitae. In some embodiments, an extract of Thuja occidentals leaf is used. In some embodiments, it provides antiviral effects. In other embodiments it is considered a booster of the immune system for its immunostimulant-like effects.

Thymic Protein A—a protein produced by the human thyroid that regulates various immune cell activity, including that of T cells. In certain embodiments, the protein can be synthetically produced as a powder. In many embodiments, it is considered a booster of the immune system.

Transfer factor—a dietary supplement commonly derived from bovine colostrum or egg yolk. In some embodiments, either source of transfer factor can be used. In some embodiments, a similar biomolecule derived from human biology can be used. In some embodiments, transfer factor is considered a booster of the immune system for its ability to improve antigen recognition.

Turkey tail mushrooms—the fungus Trametes versicolor, synonymous with Coriolus versicolor and Polyporus versicolor. In some embodiments, it provides a high concentration of antioxidants. In some embodiments, it is considered a booster of the immune system. In other embodiments, it provides anti-cancer effects. In still further embodiments, it can promote the growth of healthy gut bacteria.

Turmeric—the plant Curcuma longa. It contains high quantities of the compound curcumin. In some embodiments, it provides anti-inflammatory effects. In additional embodiments, it provides antiviral effects. In still further embodiments it is considered a booster of the immune system and can proactively immunomodulate it.

Umcka—the plant Pelargonium sidoides also known as African geranium or South African geranium. In some embodiments, it is considered a booster of the immune system. In other embodiments, it can reduce bronchitis and flu symptoms, thereby providing anti-infection effects.

Vitamin A—a group of essential unsaturated biomolecules. In some embodiments, the vitamin A is about 90% beta-carotene. In additional embodiments, the vitamin A is derived from carrots (Daucus carota subsp. Sativus). In other embodiments other proportions of the various compounds of vitamin A are used as well as other sources. In some embodiments, vitamin A maintains healthy regulation of hematopoietic stem cell development. It is considered a booster of the immune system in many embodiments.

Vitamin B12—an essential biomolecule, also known as cobalamin, that can exist as one of four variants cyanocobalamin, hydroxocobalamin, adenosylcobalamin, and methylcobalamin. In some embodiments, methylcobalamin is used. In further embodiments, the methylcobalamin is derived from yeast (S. cerevisiae). As used herein, proportional weights are calculated from methylcobalamin. In other embodiments, other variants and sources can be used. Vitamin B12 provides key metabolic resources, and in some embodiments, increases white blood cell count.

Vitamin C—a vital nutrient also known as ascorbic acid or ascorbate. In some embodiments, the vitamin C is derived from the plant Malpighia emarginata commonly known as the acerola cherry. In many embodiments, vitamin C is considered a booster of the immune system. In addition to being an antioxidant, the vitamin can increase white blood cell count in some embodiments.

Vitamin D3—a vital nutrient also known as cholecalciferol. In some embodiments, the vitamin is derived from lichens. In some embodiments, vitamin D3 is considered a booster of the immune system due to its ability to increase the production of various immune cells, such as T-cells and macrophages.

Vitamin E—a group of eight related vital nutrients including tocopherols and tocotrienols. In some embodiments, the vitamin E employed is DL-alpha-tocopheryl acetate. In some embodiments, the DL-alpha-tocopheryl acetate is derived from soy beans (Glycine max). In other embodiments other variants and sources of vitamin E can be employed. As used herein, proportional weights are calculated from DL-alpha-tocopheryl acetate. In some embodiments, vitamin E operates as an effective antioxidant, and in other embodiments, it can assist in maintaining a healthy immune system in elderly people. In some embodiments, it is considered a booster of the immune system.

Wild indigo root—roots of the plants of genus Baptisia. In some embodiments, an extract of wild indigo root is used. In some embodiments, it provides antiviral effects. In other embodiments it is considered a booster of the immune system for its immunostimulant-like effects.

Zinc—a chemical element and essential nutrient. In some embodiments, zinc is provided as a chelated zinc powder from brown rice chelate. In other embodiments, other sources of zinc can be employed. As used herein, proportional weights are calculated from chelated zinc powder from brown rice chelate. In some embodiments, zinc is considered a booster of the immune system as it can promote the development of various immune system cells such as neutrophils, macrophages and natural killer cells. In some embodiments, zinc can inhibit the replication of a virus once it has entered a cell.

### Methods

As shown in FIG. 5, one embodiment of a method 500 for treating or preventing a viral infection in a human patient includes providing doses of the above herbal compositions as well as doses of apitoxin, the primary venom in bee stings. The method functions to treat or prevent viral infections in human patients, and in some embodiments, the method functions to treat or prevent infection of COVID-19. The method is used in immunology, in particular, treating and preventing various viral infections, but can additionally or alternatively be used for any suitable applications, clinical or otherwise. The method can be configured and/or adapted to function for any suitable viral infection.

At Step 501, the method 500 optionally includes providing a dose of a fortifying herbal composition to a patient at least once a day for at least one day. This fortifying herbal composition can comprise bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12 as described in various embodiments above in FIG. 3. In further embodiments, the fortifying composition can further comprise Triphala, Andrographis paniculata, beleric myrobalan, chebulic myrobalan, papaya fruit, and Thymic Protein A as shown in FIG. 4. The dosage of the fortifying herbal composition can vary between embodiments, ranging from about 300 mg to about 9 grams. The dose of the fortifying herbal composition can be provided more than one time a day. In some embodiments, the dose of the fortifying herbal composition is provided four times a day. In further embodiments, the dose of the fortifying herbal composition is about 1000 mg and is provided four times a day for a total daily dosage of about 4000 mg. In additional embodiments, the dose of the fortifying herbal composition is about 1250 mg and is provided four times a day for a total dosage of about 5000 mg. In still further embodiments, the one or more doses of the fortifying herbal mixture is taken with food or after a meal (i.e., postprandially). In some embodiments, the one or more doses of the fortifying herbal composition can be provided each day over any number of days. In some embodiments, the one or more doses of the fortifying herbal composition can be provided each day for at least two weeks. In some embodiments, the one or more doses of the fortifying herbal composition can be provided each day for at least four weeks. In some embodiments, the fortifying herbal composition can be taken alongside and on the same day of doses of a pretreatment herbal composition, a treatment herbal composition, and/or a dose of apitoxin as described here. In some embodiments, providing a dose of the fortifying composition, alone or with other herbal compositions as described herein, can be considered as a bolstering the human immune systems against infections.

At Step 502, the method 500 optionally includes providing a dose of a pretreatment herbal composition to a patient at least once a day for at least one day. This pretreatment herbal composition can comprise Andrographis paniculata, Eleuthero roots, and resistant potato starch as described in various embodiments above in FIG. 1. The dosage of the pretreatment herbal composition can vary between embodiments, ranging from about 300 mg to about 9 grams. The dose of the pretreatment herbal composition can be provided more than one time a day. In some embodiments, the dose of the pretreatment herbal composition is provided three times a day. In further embodiments, the dose of the pretreatment herbal composition is about 1000 mg and is provided three times a day for a total daily dosage of about 3000 mg. In still further embodiments, the one or more doses of the pretreatment herbal mixture is taken with food or after a meal (i.e., postprandially). In some embodiments, the one or more doses of the pretreatment herbal composition can be provided each day over any number of days. In some embodiments, the one or more doses of the pretreatment herbal composition can be provided each day for two to seven days. In some embodiments, the one or more doses of the pretreatment herbal composition can be provided each day for four to five days. In some embodiments, step 502 comprising providing a dose of a pretreatment herbal composition comprises the only step in method 500, such that no fortifying or treatment step is required.

Next, at optional Step 504, the method 500 includes waiting at least twenty-four hours after the last dose of the pretreatment herbal mixture provided in Step 502 before proceeding to Step 506. In some embodiments, the method 500 includes waiting at least forty-eight hours after the last dose of the pretreatment herbal mixture provided in Step 502 before proceeding to Step 506. In further embodiments, the method includes waiting about forty-eight hours after the last dose of the pretreatment herbal mixture provided in Step 502 before proceeding to Step 506.

At Step 506, the method 500 includes providing a dose of apitoxin to the patient at least once a day for at least one day. Apitoxin is the venom from honeybees (genus Apis), and it contains a mixture of over sixty-three proteins and other biomolecules, including melittin and various phospholipase A2 (PLA2s). Melittin, the primary protein in apitoxin, accounting for about 50% of the dry weight of apitoxin, can combat viral infections in some embodiments by killing the virus itself, disrupting its ability to enter into mammalian cells, and reducing inflammation, which in turn improves the immune response. In some embodiments, PLA2s provide a similar anti-inflammatory effect and a similar immune bolstering response. Though it can be collected from most honeybees, in some embodiments, the apitoxin of the European Honey Bee (Apis mellifera) is used.

In many embodiments, the dose of apitoxin is provided as a parenteral injection, although other delivery means are considered and contained herein. In some embodiments, the dose of apitoxin is delivered as a subcutaneous injection. In further embodiments, the subcutaneous injections are intentionally delivered at areas of the body that the patient feels to be particularly weak or tender from the viral infection illness. In some embodiments, the apitoxin dose is prepared from dry apitoxin powder. In some embodiments, the resulting apitoxin solution in water contains 0.9% sodium chloride by dry weight percentage.

In some embodiments, the dose of apitoxin is provided each day for at least four days. In other embodiments, the dose of apitoxin is provided each day for six days. In some embodiments, the dose of apitoxin can be from about 50 to about 750 micrograms (μg) dry per day. In other embodiments, the dose of apitoxin is at least about 200 μg dry per day. In other embodiments, the dose of apitoxin can change or vary between each day of treatment. In some embodiments, the dose of apitoxin increases between at least some of the days. In some embodiments, the dose of apitoxin is about 200 μg dry for the first day, 300 μg dry for the second and third days, and 500 μg dry for the fourth and any subsequent days. There are about 100 μg of apitoxin in a standard sting of a European Honeybee.

Next at Step 508, the method 500 includes providing a dose of a glucosamine product to the patient at least once a day for at least one day. Glucosamine is an amino sugar that is a common component of many polysaccharides, including chitin. Glucosamine can often be formulated to include various salts (e.g., sulfate, hydrochloride salt, etc.) or paired with other polysaccharides or biomolecules (e.g., chondroitin, etc.). In some embodiments, glucosamine sulfate is used as the glucosamine product. In some embodiments, the glucosamine product provides pain relief and some antiviral activity to the patient.

In some embodiments, the dose of the glucosamine product can be about 250 mg to about 1500 mg. In other embodiments, the dose of the glucosamine product can be about 500 mg. In further embodiments, the dose of the glucosamine product can be provided more than one time a day. In some embodiments, an approximately 500 mg dose of a glucosamine product is provided twice a day for a total daily dose of 1000 mg. In other embodiments, the at least one dose of the glucosamine product is provided at least on each day a dose of apitoxin is provided to the patient. In still further embodiments, the dose of the glucosamine product is taken with food or after a meal (i.e., postprandially).

Next at Step 510, a dose of a treatment herbal composition is provided to a patient at least once a day for at least one day. This treatment herbal composition comprises cannabidiol, bee pollen, and coconut oil as described in various embodiments above in FIG. 2. The dosage of the treatment herbal composition can vary in certain embodiments from about 250 mg to about 1500 mg. In other embodiments, the dose of the herbal composition can be about 500 mg. In further embodiments, the dose of the treatment herbal composition can be provided more than one time a day. In some embodiments, an approximately 500 mg dose of the treatment herbal composition is provided twice a day for a total daily dose of 1000 mg. In other embodiments, the at least one dose of the treatment herbal composition is provided at least on each day a dose of apitoxin is provided to the patient. In still further embodiments, the treatment herbal composition is taken with food or after a meal (i.e., postprandially). In some embodiments, step 410 comprising providing a dose of a treatment herbal composition comprises the only step in method 400, such that no fortifying or pre-treatment step is required.

### Prophetic Example 1

The following prophetic example serves to illustrate possible embodiments of the method 500 of FIG. 5 with additional concerns towards patient population demographics. Due to the complexity of most biological pathways, not every treatment is suitable for every patient, and it can be necessary in certain embodiments to modify or forgo entire herbal compositions and/or treatment steps. A few such potential concerns are described below.

In some embodiments, the pretreatment herbal composition should not be provided to children under the age of eighteen years or to pregnant women. In some further embodiments, the pretreatment herbal composition can only be provided to those who are sick with a dangerous viral infection, such as COVID-19. In some embodiments, the pretreatment herbal composition can have negative interactions with certain long-term medications including, but not limited to, aspirin, blood thinners such as warfarin, and high blood pressure medicines. In these embodiments, it is then necessary to either adjust the patient's medication schedules or forgo the administration of the pretreatment herbal composition. In some embodiments, the patients should wait at least forty-eight hours after the cessation of their long-term medications before taking the pretreatment herbal composition. Furthermore, in some embodiments, patients should avoid consuming alcohol for the duration of the treatment.

In some embodiments, the apitoxin dose should not be provided to children of four years of age or less or to pregnant women. In some further embodiments, the pretreatment herbal composition can only be provided to those who are sick with a dangerous viral infection, such as COVID-19. Furthermore, patients undergoing the apitoxin doses should avoid consuming alcohol for the duration of their treatment in certain embodiments. However, in further embodiments, the apitoxin doses are safe for patients with known allergies to insect bites and stings, including bee stings.

In some embodiments, the treatment herbal composition should not be provided to children of four years of age or less or to pregnant women. In some further embodiments, the pretreatment herbal composition can only be provided to those who are sick with a dangerous viral infection, such as COVID-19. Furthermore, patients taking the treatment herbal composition should avoid consuming alcohol for the duration of their treatment in certain embodiments.

Finally, in some embodiments, the fortifying herbal composition should not be provided to children of four years of age or less or to pregnant women.

### Working Example 2

In Vivo Efficacy, Toxicology, and Pathology Study in SD Rats

A study in Sprague Dawley (SD) rats was undertaken to explore the effects of the fortifying herbal composition in healthy and diseased subjects. Thirty-six SD rats (eighteen male, and eighteen female) were distributed into four groups: Control (Group A), Positive control (Group B), Low dose treatment (Group C), and High dose treatment (Group D). Animals were numbered sequentially through the groups with an equal quantity of males and females in each group (see Table A below). For the first twenty days of the twenty-eight-day experiment, animals of Group C and Group D received a 1550 mg/kg and 2325 mg/kg dose, respectively, twice daily of the embodiment of the fortifying composition of FIG. 4. Doses of the fortifying composition were stirred up in a solution of 0.5% carboxymethyl cellulose in water (i.e., the vehicle) and administered orally at a volume of 10 ml/kg. Animals of Group A and Group B received only the vehicle during the first twenty days. On the twenty-first day, animals of Group B, Group C, and Group D received a dose of COVID-19 or H1N1 spike proteins. Blood samples were taken for hematological and flow cytometry analyses from a male and female animal of each group on Day 0 of the experiment. Subsequent blood samples were taken from all animals on Day 21, except for those of Group B, and again from all non-sacrificed animals, including those of Group B, on Day 28. Two animals (one male and one female) from Group A and four animals (two male and two female) from Group C and Group D each were sacrificed on Day 21, and all remaining animals were sacrificed on Day 28. When relevant, blood samples were collected before any dose was given. Blood samples were utilized for hematological and flow cytometry analysis, and the sacrificed animals were scrutinized under necropsy, gross pathology, and histopathology methodologies.

TABLE A

| Group | | | Dose | Animal No. | Animal No. |
|---|---|---|---|---|---|
| No. | Group Name | Treatment | (mg/kg) | Male | Female |
| A | Control | V | 0 | 01-02 | 03-04 |
| B | Positive | V + SP | 0 | 05-08 | 09-12 |
| | Control | | | | |
| C | Normal Dose | FC + SP | 1550 | 13-18 | 19-24 |
| D | High Dose | FC + SP | 2325 | 25-30 | 31-36 |

Abbreviations: V = vehicle, SP = spike protein, FC = fortifying composition

Hematological analysis performed before the administration of spike proteins revealed a non-significant but dose dependent reduction in white blood cell (WBC) count and increase in hemoglobin concentration (HGB) in males treated with the fortifying composition, while similarly treated female animals showed a non-significant but dose dependent increase in WBC and mean corpuscular hemoglobin concentration (MCHC). Further analysis after the administration of spike proteins revealed that they caused an increase in WBC, HGB, and hematocrit (HCT) in the controls. For animals dosed with H1N1 spike proteins, prior treatment with the fortifying composition resulted in a dose dependent reversal in WBC and HCT and a statistically significant reversal for HGB. For animals dosed with COVID-19 spike proteins, prior treatment with the fortifying composition suggested a reversal in male rats and demonstrated a drop below the levels of the controls in female rats.

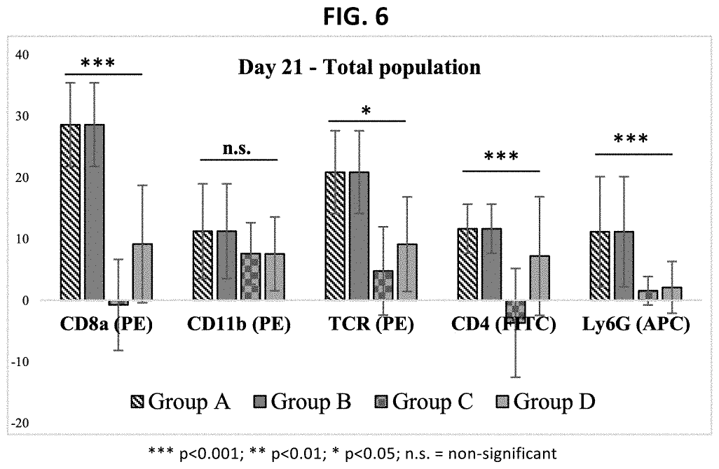

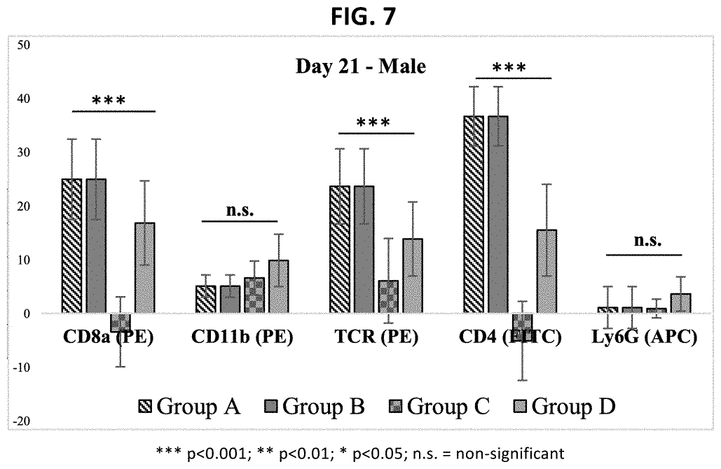

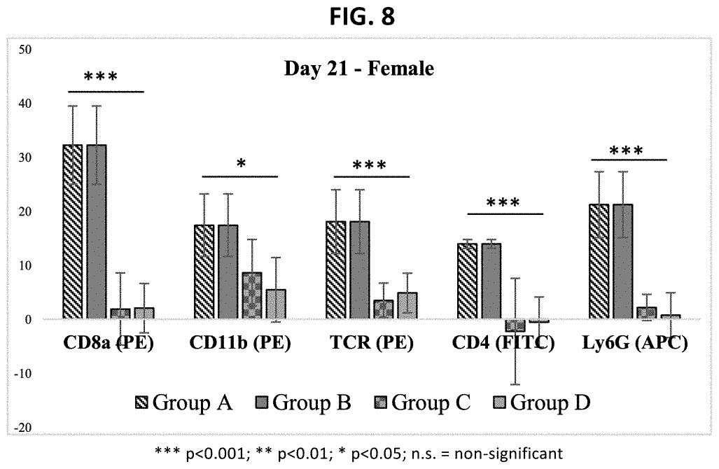

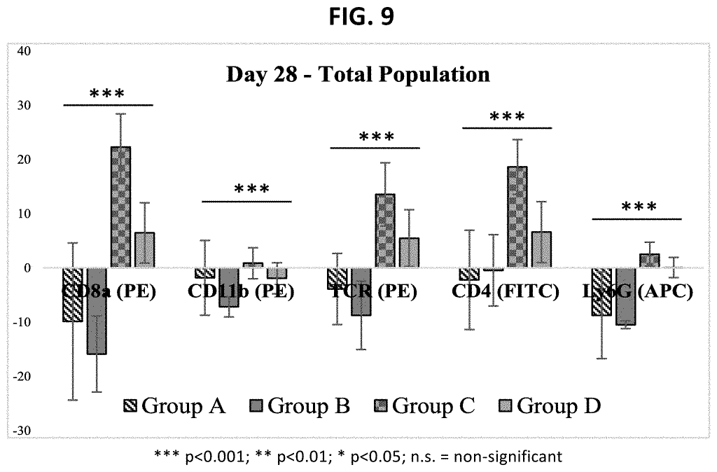

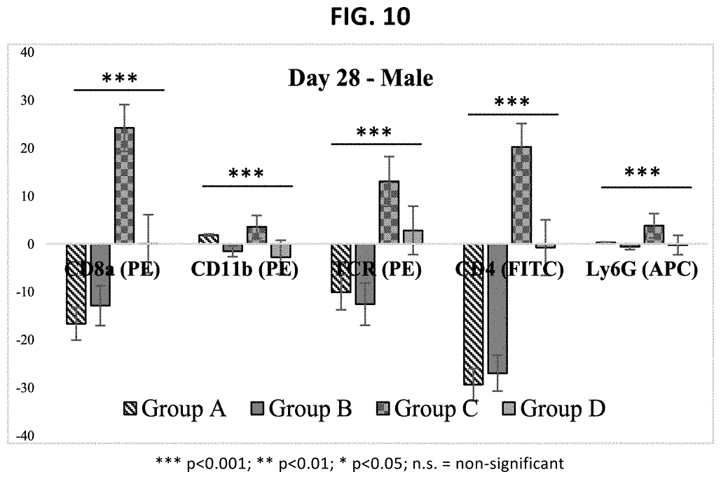

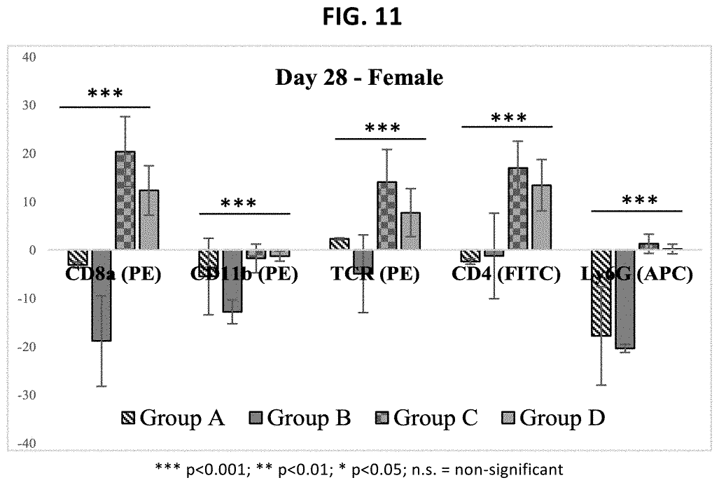

The results of flow cytometry analysis correlated with that of hematology. Blood samples adequately prepared and stained for detection of the concentration of CD8a, CD4, CD11b, TCR-alpha, Ly6G, all of which track various lymphocytes, macrophages, and granulocytes, were analyzed with a Thermofisher Attune™ Nxt Flow Cytometer, and the percentages reported herein are derived by normalizing the reading to the isotype controls. Subsequently, Day 21 data was normalized against the corresponding Day 0 data, as were Day 28 results to Day 21. FIGS. 6, 7, and 8 report the results from Day 21 samples for Total Population, Males, and Females, respectively, and FIGS. 9, 10, and 11 report the results from Day 28 samples for Total Population, Males, and Females, respectively. Results taken on Day 21 before the administration of spike proteins report greater levels of CD8a, CD4, and Ly6G and a greater expression of TCR-alpha in the control animals compared to the treatment groups, with the reduction of Ly6G more prominent and an additional reduction of CD11b in female animals. This is interpreted as a mild infection appearing in the control animals during their acclimatization in the lab that was mitigated or prevented by the fortifying composition in the treatment groups. Results from Day 28 showed that the control group returned to healthy levels while the disease control group showed a drastic decline in all markers, indicating that these animals struggled to combat the spike proteins. Conversely, all animals in the treatment groups showed an increase in all markers, indicative of a stronger immune response to the spike proteins. No dose-dependent effects were observed in this analysis.

Gross pathology and necropsy revealed COVID-19 and H1N1 spike proteins caused minimal congestion in the lungs of male disease control animals, while COVID-19 additionally produced mild red pinpoint spots in the lungs of these animals. In female disease control animals, both spike proteins generated minimal red pinpoint spots and minimal circumscribed raised white patches in the lungs. Animals with prior treatment of the fortifying composition presented dose dependent reduction of lung lesions, and results were of greater efficacy in H1N1 animals.

Histopathology revealed the mitigation and reversal of some symptoms caused by the various spike proteins. H1N1 spike proteins caused mild congestion in the alveolar septa in all animals except one male receiving the high dose treatment. Minimal focal hemorrhaging and minimal multifocal infiltration of polymorphonuclear leukocytes (PMN cells) in interalveolar septa in the lungs were observed in the H1N1 disease control group in both sexes and in some males of the treatment group, but not at all in the female treatment group animals. Moreover, perivascular edema in the lungs generated by the H1N1 spike proteins was completely absent from all treatment group animals. In other organs of H1N1 dosed animals, myocardial degeneration observed in disease control group was absent in treated animals, and lymphoid depletion in the spleen was mitigated by pretreatment with the fortifying composition low dose females and completely absent in high dose females.

In male animals of the COVID-19 disease control group, congestion in the alveolar septa, minimal multifocal alveolar macrophages, minimal focal hemorrhaging of the lungs, minimal focal emphysema, PMN infiltration into septa, and mild multifocal perivascular edema of the lungs were all observed. Pretreatment with the fortifying composition only showed reduction of symptoms in severity of PMN infiltration, perivascular edema, and quantity of alveolar macrophages at both dosages. Furthermore, pretreatment showed a dose dependent reversal in lymphoid depletion in the spleen. In female animals receiving COVID-19 spike proteins, pretreatment with the fortifying composition showed reduced severity of PMN infiltration of alveolar septa and perivascular edema and the absence of any pulmonary hemorrhaging. Female animals treated with the higher dose also exhibited mitigated congestion of the alveolar septa as well as a reduction of emphysema from mild to minimal. Lymphoid depletion in the spleen was mitigated by pretreatment with the fortifying composition, reducing the mild lymphoid depletion observed in the female disease control animals to at least minimal levels if not completely reversing it at both treatment dosages.

Overall, the various improvements of certain symptoms in SD rats in the treatment groups Group C and Group D over the controls, as detected by multiple observational metrics, suggests that the fortifying composition can have efficacy in mitigating or preventing viral infections, including, but not limited to, those of H1N1 and COVID-19.

As used in the description and claims, the singular form “a”, “an” and “the” include both singular and plural references unless the context clearly dictates otherwise. For example, the term “dose” may include, and is contemplated to include, a plurality of doses. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A fortifying herbal composition comprising: bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

2. The fortifying herbal composition of claim 1, wherein by weight percentage:
the bovine thymus, Astragalus roots, bovine red bone marrow, and turmeric are each about 5% to about 20%;
the bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%;
the beta nicotinamide mononucleotide, bovine serum albumin, Echinacea pallida roots, Echinacea purpurea, red marine algae, rosemary leaf, and Thuja occidentals leaf are each about 0.5 to about 5%;
the beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 2%; and
the lycopene, vitamin D3, vitamin A, selenium, and vitamin B12 are each about 0.0005% to about 0.3%.

3. The fortifying herbal composition of claim 1, wherein by weight percentage:
the bovine thymus, Astragalus roots, bovine red bone marrow, and turmeric are each about 5% to about 10%;
the bovine colostrum, bovine immunoglobulin, and vitamin C are each about 3% to about 8%, the turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%;
the beta nicotinamide mononucleotide, bovine serum albumin, Echinacea pallida roots, Echinacea purpurea, red marine algae, rosemary leaf, and Thuja occidentals leaf are each about 1% to about 3%;
the beta-glucan, transfer factor, umcka, vitamin E, wild indigo roots, oregano, and zinc are each about 0.3% to about 1.5%;
the lysozyme, bee pollen, melatonin, bentonite clay, royal jelly, adenosine triphosphate, LH01-myoviridae, LL12-myoviridae, LL15-Siphoviridae, and T4D-Myroviridae are each about 0.1% to about 1%; and
the lycopene, vitamin D3, vitamin A, selenium, and vitamin B12 are each about 0.0005% to about 0.1%.

4. The fortifying herbal composition of claim 1, wherein by weight percentage:
the bovine thymus is about 8.693%;
the Astragalus roots are about 8.196%;
the bovine red bone marrow is about 8.072%;
the turmeric is about 7.451%;
the bovine colostrum is about 5.588%;
the bovine immunoglobulin and vitamin C are each about 4.967%;
the turkey tail mushrooms are about 3.726%;
the Eleuthera roots and Sambucus nigra are each about 3.105%;
the cats claw bark, lactoferrin, licorice, and the olive leaf are each about 2.484%;
the Adhatoda vasica, ashwagandha, bee glue, reishi mushrooms, and shiitake mushrooms are each about 2.235%;
the rose hip is about 1.987%;
the arabinogalactan is about 1.739%;
the beta nicotinamide mononucleotide, bovine serum albumin, Echinacea pallida roots, Echinacea purpurea, red marine algae, rosemary leaf, and Thuja occidentals leaf are each about 1.242%;
the beta-glucan and transfer factor are each about 0.993%;
the umcka, vitamin E, and wild indigo roots are each about 0.745%;
the oregano and zinc are each about 0.621%;
the lysozyme is about 0.497%;
the bee pollen is about 0.373%;
the melatonin is about 0.298%;
the bentonite clay and royal jelly are each about 0.248%;
the adenosine triphosphate, LH01-Myoviridae, LL12-Myoviridae, LL15-Siphoviridae, and T4D-Myroviridae are each about 0.124%;
the lycopene is about 0.099%;
the vitamin D3 is about 0.072%;
the vitamin A is about 0.025%;
the selenium is about 0.005%; and
the vitamin B12 is about 0.001%.

5. The fortifying herbal composition of claim 1, further comprising Triphala, Andrographis paniculata, beleric myrobalan, chebulic myrobalan, papaya fruit, and Thymic Protein A.

6. The fortifying herbal composition of claim 5, wherein by weight percentage:
the Triphala, bovine thymus, Astragalus roots, bovine red bone marrow, and turmeric are each about 5% to about 20%;
the Andrographis paniculata, bovine colostrum, bovine immunoglobulin, vitamin C, turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%;
the beta nicotinamide mononucleotide, bovine serum albumin, Echinacea pallida roots, Echinacea purpurea, papaya fruit, red marine algae, rosemary leaf, and Thuja occidentals leaf are each about 0.5 to about 5%;
the beta-glucan, transfer factor, umcka, vitamin E, wild indigo roots, oregano, zinc, lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 2%;
the lycopene, vitamin D3, vitamin A, selenium, and vitamin B12 are each about 0.0005% to about 0.3%; and
the Thymic Protein A is about 0.0001% to about 0.3%.

7. The fortifying herbal composition of claim 5, wherein by weight percentage:
the Triphala is about 5% to about 15%;
the bovine thymus, Astragalus roots, bovine red bone marrow, and turmeric are each about 5% to about 10%;
the Andrographis paniculata, bovine colostrum, bovine immunoglobulin, and vitamin C are each about 3% to about 8%;
the turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%;
the beta nicotinamide mononucleotide, bovine serum albumin, Echinacea pallida roots, Echinacea purpurea, papaya fruit, red marine algae, rosemary leaf, and Thuja occidentals leaf are each about 1% to about 3%;
the beta-glucan, transfer factor, umcka, vitamin E, wild indigo roots, oregano, and zinc are each about 0.3% to about 1.5%;
the lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 1%;
the lycopene, vitamin D3, vitamin A, selenium, and vitamin B12 are each about 0.0005% to about 0.1%;
and the Thymic Protein A is about 0.0001% to about 0.1%.

8. The fortifying herbal composition of claim 5, wherein by weight percentage:
the Triphala is about 9.9%;
the bovine thymus is about 7.0%;
the Astragalus roots is about 6.6%;
the bovine red bone marrow is about 6.5%;
the turmeric is about 6.0%;
the Andrographis panticulata is about 5.0%;
the bovine colostrum is about 4.5%;
the bovine immunoglobulin and vitamin C are each about 4.0%;
the turkey tail mushrooms are about 3.0%;
the Eleuthera roots and Sambucus nigra are each about 2.5%;
the beleric myrobalan, chebulic myrobalan, cats claw bark, lactoferrin, licorice, and olive leaf are each about 2.0%;
the Adhatoda vasica, ashwagandha, bee glue, reishi mushrooms, and shiitake mushrooms are each about 1.8%;
the rose hip is about 1.6%;
the arabinogalactan is about 1.4%;
the beta nicotinamide mononucleotide, bovine serum albumin, Echinacea pallida roots, Echinacea purpurea, papaya fruit, red marine algae, rosemary leaf, and Thuja occidentals leaf are each about 1.0%;
the beta-glucan and transfer factor are each about 0.8%;
the umcka, vitamin E, and wild indigo root are each about 0.6%;
the oregano and zinc are each about 0.5%;
the lysozyme is about 0.4%;
the bee pollen is about 0.3%;
the melatonin is about 0.24%;
the bentonite clay and royal jelly are each about 0.2%;
the adenosine triphosphate, LH01-myoviridae, LL12-myroviridae, LL15-Siphoviridae, and T4D-myroviridae are each about 0.1%;
the lycopene is about 0.08%;
the vitamin D3 is about 0.058%;
the vitamin A is about 0.022%;
the selenium is about 0.004%;
the vitamin B12 is about 0.001%; and
the Thymic Protein A is about 0.00024%.

9. A method for bolstering the human immune system of a patient against infections comprising:
providing a dose of a fortifying composition at least once daily for at least one day;
wherein the fortifying composition comprises bovine thymus, Astragalus roots, bovine red bone marrow, turmeric, bovine colostrum, bovine immunoglobulin, vitamin C, turkey tail mushrooms, Eleuthera roots, Sambucus nigra, cats claw bark, lactoferrin, licorice, olive leaf, Adhatoda vasica, ashwagandha, bee glue, reishi mushrooms, shiitake mushrooms, rose hip, arabinogalactan, beta nicotinamide mononucleotide, bovine serum albumin, Echinacea pallida roots, Echinacea purpurea, red marine algae, rosemary leaf, Thuja occidentals leaf, beta-glucan, transfer factor, umcka, vitamin E, wild indigo roots, oregano, zinc, lysozyme, bee pollen, melatonin, bentonite clay, royal jelly, adenosine triphosphate, LH01-myoviridae, LL12-myoviridae, LL15-Siphoviridae, T4D-Myroviridae, lycopene, vitamin D3, vitamin A, selenium, and vitamin B12.

10. The method of claim 9, wherein of the fortifying composition, by weight percentage:
the bovine thymus is about 8.693%;
the Astragalus roots are about 8.196%;
the bovine red bone marrow is about 8.072%;
the turmeric is about 7.451%;
the bovine colostrum is about 5.588%;
the bovine immunoglobulin and vitamin C are each about 4.967%;
the turkey tail mushrooms are about 3.726%;
the Eleuthera roots and Sambucus nigra are each about 3.105%;
the cats claw bark, lactoferrin, licorice, and the olive leaf are each about 2.484%;
the Adhatoda vasica, ashwagandha, bee glue, reishi mushrooms, and shiitake mushrooms are each about 2.235%;
the rose hip is about 1.987%;
the arabinogalactan is about 1.739%;
the beta nicotinamide mononucleotide, bovine serum albumin, Echinacea pallida roots, Echinacea purpurea, red marine algae, rosemary leaf, and Thuja occidentals leaf are each about 1.242%;
the beta-glucan and transfer factor are each about 0.993%;
the umcka, vitamin E, and wild indigo roots are each about 0.745%;
the oregano and zinc are each about 0.621%;
the lysozyme is about 0.497%;
the bee pollen is about 0.373%;
the melatonin is about 0.298%;
the bentonite clay and royal jelly are each about 0.248%;
the adenosine triphosphate, LH01-Myoviridae, LL12-Myoviridae, LL15-Siphoviridae, and T4D-Myroviridae are each about 0.124%;
the lycopene is about 0.099%;
the vitamin D3 is about 0.072%;
the vitamin A is about 0.025%;
the selenium is about 0.005%; and
the vitamin B12 is about 0.001%.

11. The method of claim 9, wherein the fortifying composition further comprises Triphala, Andrographis paniculata, beleric myrobalan, chebulic myrobalan, papaya fruit, and Thymic Protein A.

12. The method of claim 9, wherein of the fortifying composition, by weight percentage:
the Triphala, bovine thymus, Astragalus roots, bovine red bone marrow, and turmeric are each about 5% to about 20%;
the Andrographis paniculata, bovine colostrum, bovine immunoglobulin, vitamin C, turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 7%;
the beta nicotinamide mononucleotide, bovine serum albumin, Echinacea pallida roots, Echinacea purpurea, papaya fruit, red marine algae, rosemary leaf, and Thuja occidentals leaf are each about 0.5 to about 5%;
the beta-glucan, transfer factor, umcka, vitamin E, wild indigo roots, oregano, zinc, lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 2%;
the lycopene, vitamin D3, vitamin A, selenium, and vitamin B12 are each about 0.0005% to about 0.3%; and
the Thymic Protein A is about 0.0001% to about 0.3%.

13. The method of claim 9, wherein of the fortifying composition, by weight percentage:
the Triphala is about 5% to about 15%;
the bovine thymus, Astragalus roots, bovine red bone marrow, and turmeric are each about 5% to about 10%;
the Andrographis paniculata, bovine colostrum, bovine immunoglobulin, and vitamin C are each about 3% to about 8%;
the turkey tail mushrooms, Eleuthera roots, Sambucus nigra, beleric myrobalan, chebulic myrobalan, cats claw bark, lactoferrin, licorice, olive leaf, Adhatoda vasica, ashwagandha, bee glue, reishi mushrooms, shiitake mushrooms, rose hip, and arabinogalactan are each about 1% to about 5%;
the beta nicotinamide mononucleotide, bovine serum albumin, Echinacea pallida roots, Echinacea purpurea, papaya fruit, red marine algae, rosemary leaf, and Thuja occidentals leaf are each about 1% to about 3%;
the beta-glucan, transfer factor, umcka, vitamin E, wild indigo roots, oregano, and zinc are each about 0.3% to about 1.5%;
the lysozyme, bee pollen, melatonin, bentonite clay, royal jelly, adenosine triphosphate, LH01-Myoviridae, LL12-Myoviridae, LL15-Siphoviridae, and T4D-Myroviridae are each about 0.1% to about 1%;
the lycopene, vitamin D3, vitamin A, selenium, and vitamin B12 are each about 0.0005% to about 0.1%; and
the Thymic Protein A is about 0.0001% to about 0.1%.

14. The method of claim 9, wherein of the fortifying composition, by weight percentage:
the Triphala is about 9.9%;
the bovine thymus is about 7.0%;
the Astragalus roots is about 6.6%;
the bovine red bone marrow is about 6.5%;
the turmeric is about 6.0%;
the Andrographis panticulata is about 5.0%;
the bovine colostrum is about 4.5%;
the bovine immunoglobulin and vitamin C are each about 4.0%;
the turkey tail mushrooms are about 3.0%;
the Eleuthera roots and Sambucus nigra are each about 2.5%;
the beleric myrobalan, chebulic myrobalan, cats claw bark, lactoferrin, licorice, and olive leaf are each about 2.0%;
the Adhatoda vasica, ashwagandha, bee glue, reishi mushrooms, and shiitake mushrooms are each about 1.8%;
the rose hip is about 1.6%;
the arabinogalactan is about 1.4%;
the beta nicotinamide mononucleotide, bovine serum albumin, Echinacea pallida roots, Echinacea purpurea, papaya fruit, red marine algae, rosemary leaf, and Thuja occidentals leaf are each about 1.0%;
the beta-glucan and transfer factor are each about 0.8%;
the umcka, vitamin E, and wild indigo root are each about 0.6%;
the oregano and zinc are each about 0.5%;
the lysozyme is about 0.4%;
the bee pollen is about 0.3%;
the melatonin is about 0.24%;
the bentonite clay and royal jelly are each about 0.2%;
the adenosine triphosphate, LH01-myoviridae, LL12-myroviridae, LL15-Siphoviridae, and T4D-myroviridae are each about 0.1%;
the lycopene is about 0.08%;
the vitamin D3 is about 0.058%;
the vitamin A is about 0.022%;
the selenium is about 0.004%;
the vitamin B12 is about 0.001%; and
the Thymic Protein A is about 0.00024%.

15. The method of claim 9, wherein the dose is about 1000 mg, and wherein the dose is provided four times daily.

16. The method of claim 9, wherein the dose is about 1250 mg, and wherein the dose is provided four times daily.

17. The method of claim 9, wherein the dose is provided at least once a day for at least fourteen consecutive days.

18. The method of claim 9, wherein after providing the dose of the fortifying composition, at least one of the markers comprising: CD8a, CD4, CD11b, TCR-alpha, or Ly6G is present at an increased level in the patient following exposure to an infection.

19. The method of claim 18, wherein at least two of the markers comprising: CD8a, CD4, CD11b, TCR-alpha, or Ly6G are present at an increased level in the patient following exposure to an infection.

20. The method of claim 19, wherein all of the markers comprising: CD8a, CD4, CD11b, TCR-alpha, or Ly6G are present at an increased level in the patient following exposure to an infection.
